(19) United States
(12) Patent Application Publication
(10) Pub. No.: US 2025/0276287 A1
(43) Pub. Date: Sep. 4, 2025

(54) SYSTEMS AND METHODS FOR ORGANIC ACID RECOVERY

(71) Applicant: Alliance for Sustainable Energy, LLC, Golden, CO (US)

(72) Inventors: Yian CHEN, Golden, CO (US); Eric M. KARP, Denver, CO (US); Gregg Tyler BECKHAM, Golden, CO (US)

(21) Appl. No.: 19/067,377

(22) Filed: Feb. 28, 2025

Related U.S. Application Data
(60) Provisional application No. 63/559,321, filed on Feb. 29, 2024.

(51) Int. Cl. B01D 61/02; B01D 3/14; B01D 11/04; B01D 61/08; B01D 69/02; B01D 69/12; B01D 71/56; C07C 51/48

(52) U.S. Cl. CPC B01D 61/025; B01D 3/145; B01D 11/0415; B01D 11/0488; B01D 61/08; B01D 69/02; B01D 69/12; B01D 71/56; C07C 51/48; B01D 2325/0283; B01D 2325/02834

(57) ABSTRACT

This disclosure provides an optimized, energy-efficient, and sustainable solution for continuous in situ bio-based chemical recovery by integrating high pressure reverse osmosis with extraction processes. These systems achieve higher recovery efficiency while reducing production costs and environmental impact.

### CROSS REFERENCE TO RELATED APPLICATION

This application claims the benefit of U.S. Provisional Application No. 63/559,321 filed Feb. 28, 2024, which is incorporated by reference herein in its entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### FIELD OF THE INVENTION

This disclosure relates to downstream processing methods and systems for recovering organic compounds, such as organic acids and biofuels, from biosynthesis reaction media. More particularly, these methods utilize high-pressure reverse osmosis (HPRO) filtration and/or biochemical extraction processes for enhanced separation efficiency, reduced energy consumption, and improved solvent recovery.

### BACKGROUND

Downstream bioprocessing (DSP) is critical in the production of bioproducts synthesized in biosynthesis processes, such as fermentation and cell-free processes. However, the complex nature of these biosystems often leads to dilute broths of low product titers with the presence of multiple impurities, making bioproduct recovery a major technical hurdle. Towards improved separation processes in bioprocessing, continuous DSP has received substantial attention due to its potential to considerably reduce production costs. As an example of continuous DSP, in situ product recovery (ISPR) aims to continuously recover bioproducts during continuous synthesis processes, such as fermentation. By continuously removing the bioproducts from the bioreactor, ISPR can mitigate product inhibition, potentially improve production rate and apparent titer, and enable continuous fermentations. Conventional separation processes, such as distillation and solvent extraction, are often energy- and chemical-intensive, leading to inefficiencies. Membrane-based separations, offer viable alternatives by concentrating target compounds while minimizing energy input and solvent usage.

For many organic chemical products produced in biosynthesis methods, solvent-based liquid-liquid extraction (LLE) is among the most well studied approach for ISPR due to its ability to achieve high product recovery yields. A solvent-based in situ LLE may be conducted with a solvent overlay in a bioreactor to continuously extract the organic compounds produced in the biosynthesis reaction. But solvent-based liquid-liquid extraction is limited by emulsion formation between immiscible aqueous and organic phases, which impairs phase separation. Additionally, solvent droplets dispersed in the aqueous phase can be toxic to production microbes in microbial fermentation processes, and water droplets dispersed in the organic phase can increase energy consumption during subsequent product purification and solvent recovery steps. Within biosynthetic schemes that include microbial fermentation processes, the toxic effects of solvents in the culture may be mitigated by a cell retention and broth clarification step before solvent extraction thereby physically retaining microbial cells in the bioreactor and preventing their direct contact with the organic solvent. A membrane contactor, in which a hydrophobic porous membrane serves as a physical barrier between the aqueous and organic phases, is typically used downstream of the cell retention unit operation for continuous solvent extraction and to prevent emulsion formation. In previously reported ISPR processes, a membrane contactor enabled continuous carboxylic acid extraction from fermentation (D. Salvachua, et al., Process intensification for the biological production of the fuel precursor butyric acid from biomass. Cell Reports Physical Science, 2021, 2, 100587; P. O. Saboe, et al., Green Chemistry, 2018, 20, 1791-1804; A. Gössi, et al., Separation & purification technology, 2020, 241, 116694; G. Burgé, et al., Journal of Chemical Technology & Biotechnology, 2016, 91, 2705-2712; S.-T. Yang, et al., Bioprocessing for value-added products from renewable resources, Elsevier, 2007, pp. 421-446; G. Burge, et al., IN SITU EXTRACTION OF 3-HYDROXYPROPIONIC ACID ASSISTED BY MEMBRANE CONTACTOR. 4th International Congress on Green Process Engineering (GPE4), April 2014, Sevilla, Spain). In the test system using an aliphatic monocarboxylic acid, butyric acid, the reported acid flux from mock fermentation broth using a membrane contactor was only 0.26 g/m²/h (at pH 5.5) with Cyanex 923 as the organic phase. The acid flux was quite limited because the aqueous and organic phases were only in contact in the membrane contactor pores, and thus a large membrane area was needed to compensate the low throughput of butyric acid extraction.

The methods of the present disclosure provide substantially improved systems and methods for recovering bioproducts from reaction media, such as fermentation broth, in continuous or batch biosynthesis processes. These methods and systems achieve higher recovery efficiency while reducing production costs and environmental impact.

### SUMMARY

An integrated system and method have been developed for recovering bioproducts, such as biofuels and organic acids, from biosynthesis processes, such as cell free reaction media or fermentation broths. The method involves pre-concentrating organic compounds from biosynthesis processes using high pressure reverse osmosis (HPRO), followed by selective extraction of the organic compounds from the concentrated HPRO retentate. These methods achieve higher efficiency recovery while reducing production costs and environmental impact.

This disclosure demonstrates the integration of high-pressure reverse osmosis (HPRO) to concentrate the bioproducts prior to downstream extraction significantly improves the maximum achievable concentration factor (CF) compared to the CF observed using conventional reverse osmosis (RO) membranes. The resulting concentrated aqueous stream undergoes extraction with an in situ product recovery (ISPR) process with greater than 5-fold increased final compound concentration in the organic phase, and a concomitant 76% reduction in organic solvent usage. These improvements can lead to substantial reductions in production cost and greenhouse gas (GHG) emissions, considerably exceeding the process performance when integrating conventional RO product concentration.

This disclosure also demonstrates a hydrophobic polytetrafluoroethylene (PTFE) membrane-based emulsion separator (MBES) enables continuous liquid-liquid extraction (LLE) for the recovery of organic fermentation products, such as biofuel and/or organic acids, with substantially improved throughput relative to a membrane contactor. Process modeling estimates that MBES-assisted LLE can enable a 66% and 40% reduction in process costs (the sum of CAPEX and OPEX) and greenhouse gas emissions, respectively, compared to previously used membrane contactors, due to an approximately 160-fold decrease in the required membrane area. Although membrane fouling from cellular debris may lead to reduced membrane flux and phase separation efficiency, common fouling mitigation strategies, including the incorporation of pre-filtration and periodic membrane backwashing, can effectively recover membrane performance.

Overall, the methods and systems of this disclosure enable continuous ISPR processes in bioprocessing separations, including where emulsion formation is a challenge, and the integration of an HPRO membrane for product concentration enables more economical and sustainable biosynthesis product recovery.

In a first aspect, this disclosure provides methods extracting organic compounds from a media containing the organic compounds and other components of the biosynthesis reactions that were used to produce the organic compounds. These methods include filtering an aqueous media containing an organic compound through a high-pressure reverse osmosis (HPRO) membrane to concentrate the organic compound in an aqueous HPRO filtration retentate. The organic compound is then extracted from the aqueous HPRO filtration retentate by a recovery process. Useful recovery processes may include any one or more of liquid-liquid extraction, distillation, crystallization, electrodialysis, chromatography, adsorption, evaporation, and combinations of these recovery processes, best suited to isolate and/or purify the organic compound. In these methods, the organic compound may be any organic compound synthesized in a biosynthesis process. For example, the organic compound may be synthesized in a bioreactor in which the synthesis reactions take place in one or more cell-free chemical reactions, or in a cellular synthesis process (i.e., a microbial fermentation biosynthesis process). In these methods, the organic compound may be a compound within any one of the chemical class of alkanes, alkenes, alkynes, aromatic hydrocarbons, alcohols, phenols, ethers, aldehydes, ketones, carboxylic acids, esters, amides, acid anhydrides, amines, nitriles, alkyl halides, aryl halides, thiols, sulfides, sulfonic acids, phosphines, phosphates, and combinations thereof. In exemplary embodiments, the organic compound may be an organic acid, such as acetic acid, propionic acid, butyric acid, valeric acid, caproic acid, lactic acid, and combinations thereof.

In the initial filtration through an HPRO membrane, the membrane may operate at a pressure between 800 psi and 1800 psi, and this filtration preferably achieves a concentration factor (CF) between 3.5 and 5.0 for the organic compound. In this HPRO filtration, the membrane may include a thin-film composite polyamide membrane.

In these methods, the extraction of the organic compound from the aqueous HPRO filtration retentate may include liquid-liquid extraction methods to produce an organic solvent containing the organic compound. The liquid-liquid extraction may be a membrane-based liquid-liquid extraction (MBLLE) that includes a membrane material such as polyethylene, high density polyethylene, perfluoroalkylate, polytetrafluoroethylene, ultra-high molecular weight polyethylene, a copolymer of ethylene and tetrafluoroethylene, polyphthalamide, polyamide, polyester, polysulfone, polyethersulfone, cellulose, cellulose acetate, regenerated cellulose, and combinations thereof. Preferably, the MBLLE membrane material has a pore size of less than 2 μm, or less than 1 μm, or between 2 μm and 0.1 μm, or between 1 μm and 0.2 μm. The liquid-liquid extraction may extract the organic compound into an organic phase with a solvent partition coefficient of at least 2.0 and/or extracts the organic compound into an organic phase with an extraction efficiency of at least 65%. In these methods, the organic compound may be isolated from the organic solvent by distillation. The distillation may purify the organic compound from the organic solvent at a purity of at least 90%.

In these methods, extracting the organic compound from the aqueous HPRO filtration retentate produces a spent HPRO retentate comprising a bioreactor reaction media. These methods may include recycling the bioreactor reaction media by returning the spent HPRO retentate to a bioreactor. In this way, the organic compound may be continuously removed from the reaction media in a continuous biosynthetic production process.

In another aspect, this disclosure provides systems for efficiently performing the methods of the first aspect of this disclosure to extract an organic compound from a media containing the organic compound. These systems include a high-pressure reverse osmosis (HPRO) unit configured to accept an aqueous stream comprising an organic compound from a bioreactor and concentrate the organic compound in an aqueous HPRO filtration retentate. Within these systems, an extraction unit is fluidly connected to the HPRO unit and is configured to isolate the organic compound from the aqueous HPRO filtration retentate to form an isolated organic compound and a spent aqueous HPRO filtration retentate. In these systems, the extraction unit includes a recovery process that may include any one or more of liquid-liquid extraction, distillation, crystallization, electrodialysis, chromatography, adsorption, evaporation, and combinations thereof. In these systems, the HPRO unit includes an HPRO membrane that operates at a pressure between 800 psi and 1800 psi, and achieves a concentration factor between 3.5 and 5.0 for the organic compound. In an embodiment of these systems the extraction unit includes a membrane-based liquid-liquid extraction (MBLLE) unit which includes a membrane material selected from polyethylene, high density polyethylene, perfluoroalkylate, polytetrafluoroethylene, ultra-high molecular weight polyethylene, a copolymer of ethylene and tetrafluoroethylene, polyphthalamide, polyamide, polyester, polysulfone, polyethersulfone, cellulose, cellulose acetate, regenerated cellulose, and combinations thereof. In this embodiment, the system may include a first recirculation system for recycling spent aqueous HPRO filtration retentate from the MBLLE unit to the bioreactor. Similarly, these systems may additionally include a second recirculation system for recycling an organic solvent from the extraction unit to be mixed with the HPRO filtration retentate.

This Summary is neither intended nor should it be construed as representative of the full extent and scope of the present disclosure. Moreover, references made herein to “the present disclosure,” or aspects thereof, should be understood to mean certain embodiments of the present invention and should not necessarily be construed as limiting all embodiments to a particular description. The present invention is set forth in various levels of detail in this Summary as well as in the attached drawings and the Detailed Description and no limitation as to the scope of the present invention is intended by either the inclusion or non-inclusion of elements, components, etc. in this Summary. Additional aspects of the present invention will become more readily apparent from the Detailed Description, particularly when taken together with the figures.

### BRIEF DESCRIPTION OF THE DRAWINGS

Exemplary embodiments are illustrated in the drawing. It is intended that the embodiments and figures disclosed herein are to be considered illustrative rather than limiting.

FIG. 1 is a schematic of one exemplary embodiment of the filtration and extraction systems of this disclosure.

FIG. 2. is a schematic diagram of the cross-flow membrane channel for a continuous product concentration process with: vertical (top) and horizontal (bottom) concentration polarization.

FIGS. 3A and 3B. Shows the RO membrane product concentration model visualization and assessment of the impacts of major factors on the maximum product CF via a set base scenario and control studies. FIG. 3A shows the major factors, including Rₘ and ΔP, and FIG. 3B shows factors k, B, and R_{fouling}. Note that FIG. 3B is the zoom in view of the small fraction of FIG. 3A circled by the dashed line.

FIGS. 4A and 4B show the perm-selective performance of commercial RO and HPRO membranes. FIG. 4A shows the performance in terms of butyric acid selectivity (A/B) as a function of water permeability coefficient (A). FIG. 4B shows the performance of water permeability coefficient, and observed and intrinsic butyric acid rejections.

FIGS. 5A-5F shows butyric acid concentrations and recoveries from C. tyrobutyricum fermentation broth using selected RO and HPRO membranes. Membrane performance during the acid concentration tests were evaluated in terms of (FIG. 5A) normalized permeate flux profile, (FIG. 5B) normalized permeate concentration profile, (FIG. 5C) butyric acid concentration factor (CF), product recovery (PR), and permeate volume recovery (Y), (FIG. 5D) tradeoff between CF and PR, (FIG. 5E) percentage contribution of membrane fouling to concentration test permeate flux decline, and (FIG. 5F) fermentation broth compositional change before and after the HPRO membrane concentration test.

FIGS. 6A and 6B show RO and HPRO membrane concentration of the various organic components in the C. tyrobutyricum fermentation broth in terms of observed membrane rejection (FIG. 6A), and concentration factor (FIG. 6B).

FIGS. 7A-7F show the impacts of RO and HPRO concentration unit operation on organic solvent extraction efficiency, as indicated by (FIG. 7A) composition of the aqueous streams before LLE (excluding water), (FIG. 7B) composition of the organic streams after LLE (excluding organic solvent), (FIG. 7C) butyric acid concentrations in the aqueous streams before LLE, (FIG. 7D) butyric acid concentrations in the organic streams after LLE, (FIG. 7E) butyric acid partition coefficients, and (FIG. 7F) butyric acid extraction efficiencies.

FIGS. 8A-8D show the continuous membrane demulsification using MBES with an effective total membrane area of 60 cm². Mock fermentation broth (10 g/L butyric acid, pH 5) and organic solvent (70 vol % Cyanex 923, 30 vol % mineral oil) were vigorously mixed using a magnetic stir plate to form an emulsion for the MBES extraction step, and then continuously separated via MBES into pure aqueous and organic phases. The extraction efficiency of butyric acid into the organic solvent was characterized using HPLC of the aqueous phase before and after the MBES unit, and the phase separation efficiency was measured using Karl Fisher titration measurement results of both the organic and aqueous phases after the MBES unit. FIG. 8A shows images and Karl Fisher titration measurements of water content in the organic solvent phase (values at bottom) after different settling time of an emulsion that is kinetically stable for 1,500 min. FIG. 8B shows the impact of the aqueous:organic phase volume ratio and permeate flux on MBES phase separation efficiency (PS). The thermodynamic limits of PS (due to the water partition coefficient) at an aqueous:organic phase volume ratio value of 0.5, 1.0, 2.0, and 3.0 are 90.5%, 93.1%, 94.9%, and 95.8%, respectively. FIG. 8C shows the membrane phase separation efficiency reversibility as indicated by the phase separation efficiency at different permeate flux (at an exemplary aqueous:organic phase volume ratio of 0.5; dark curve-direction of increasing permeate flux; light curve-direction of decreasing permeate flux). FIG. 8D shows the membrane phase separation efficiency reversibility as indicated by photos of the MBES permeate (left) and retentate (right) samples. (The error bars represent the standard deviation from at least 3 replicate measurements.)

FIGS. 9A and 9B show the fouling propensities of the hydrophobic PTFE membrane during a 1-hour filtration of both emulsified mock broth and emulsified fermentation broth as indicated by the permeate flux profile normalized to initial permeate flux of the pristine membrane (FIG. 9A) and the final water content in the permeate and retentate streams measured using Karl Fisher titration (FIG. 9B).

FIGS. 10A-10F show the energy-dispersive X-ray spectroscopy analysis (FIGS. 10A, 10C, 10E) and scanning electron microscope images (FIGS. 10B, 10D, 10F) of the surfaces of the pristine hydrophobic PTFE membrane (FIGS. 10A, 10B), and the hydrophobic PTFE membranes filtered with emulsified mock broth (FIGS. 10C, 10D) and emulsified fermentation broth (FIGS. 10E, 10F).

FIG. 11A-11D show pre-filtration of the raw Clostridium tyrobutyricum fermentation broth using both microfiltration (MF) and ultrafiltration (UF) membranes with different pore sizes can effectively reduce the average solute particle size (FIG. 11A) and remove proteins above 14 kDa (FIG. 11B), thus leading to reduced hydrophobic PTFE membrane fouling propensity (FIG. 11C) and enhanced cleaning efficacy (FIG. 11D). Membrane fouling propensity was evaluated with 1 hour filtration of the emulsified fermentation broth, and membrane cleaning efficacy was assessed with a two-minute membrane organic phase backwash with a flow rate of approximately 20 mL/min).

### DETAILED DESCRIPTION

Particular aspects of this disclosure are described in greater detail below. The terms and definitions used in the present application and as clarified herein are intended to represent the meaning within the present disclosure.

In this disclosure, the singular forms “a,” “an,” and “the” include plural reference unless the context dictates otherwise.

The terms “approximately”, “approximately”, and “about” mean nearly the same as a referenced number or value. As used herein, the terms “approximately” and “about” should be generally understood to encompass ±30%, preferably ±20%, particularly preferred ±10% and especially preferred ±5% of a specified amount, frequency or value.

The term “organic compound” as used in this disclosure includes hydrocarbons (i.e., compounds containing only carbon and hydrogen), alkanes, alkenes, alkynes, aromatic hydrocarbons, alcohols, phenols, ethers, aldehydes, ketones, carboxylic acids, esters, amides, acid anhydrides, amines, nitriles, alkyl halides, aryl halides, thiols, sulfides, sulfonic acids, phosphines, phosphates, diols, terpenes, fatty acids, nucleic acids, and combinations thereof.

The term “biosynthetic process” as used in this disclosure means a process in which living organisms produce complex organic compounds from simpler precursors. These processes typically require energy input (usually in the form of ATP) to build organic compounds. Exemplary biosynthetic processes include the formation of proteins from amino acids via ribosomes and mRNA; the synthesis of nucleic acids using nucleotide building blocks; the production of fatty acids and phospholipids; the formation of glucose and polysaccharides like glycogen or cellulose; the synthesis of steroid hormones, insulin, and other signaling molecules; and photosynthesis (in plants and cyanobacteria; i.e., the conversion of light energy into organic molecules like glucose).

The term “media” as used in this disclosure means a liquid that may include an organic compound of this disclosure. The media may support a biosynthesis process for producing an organic compound (i.e., a reaction media). In this disclosure, the media, including a reaction media, is typically an aqueous media (i.e., containing water, and further may comprise hydrophilic or hydrophobic molecules that are miscible, partially miscible, or immiscible in water). An exemplary reaction media is a fermentation media that supports the microbial fermentation processes that produce organic compounds.

The term “isolate” as used in this disclosure refers to the process of obtaining a specific compound or chemical entity from a mixture, media, reaction media, fermentation media, or biological source. Following isolation, the compound may still contain impurities, but it is separated from other substances in the media and typically is present at a higher concentration than the original media. In isolating natural products, the product is first separated from its source material, which may involve extracting it from a plant, microorganism, or other biological source. An example of isolating a synthesized compound would include synthesizing an organic compound in a reaction media and removing it from the reaction mixture.

The terms “purify” and “purification” as used in this disclosure refer to refining the isolated compound to increase its purity, removing contaminants, byproducts, or unwanted substances, such that the concentration and homogeneity of the compound is increased. Examples of purification include using chromatography, crystallization, or recrystallization to enhance the purity of a previously isolated organic compound. Purification often results in specific levels of purity (e.g., >95% pure).

The present disclosure describes a process for extracting an organic compound from a media, particularly an aqueous media, by the steps of filtration and/or extraction. In some embodiments of the present disclosure, the filtration step may include a high-pressure reverse osmosis (HPRO) filtration of the media to concentrate the organic compound in the media. The subsequent extraction step may include one or more of liquid-liquid extraction, distillation, crystallization, electrodialysis, chromatography, adsorption, evaporation, or combinations of these techniques to isolate and/or purify the organic compound from the media. Thus, the methods and systems of the present disclosure provide methods for isolating organic compounds, which effectively recovers the organic compound at a higher concentration from a low-concentration aqueous media.

Methods of this Disclosure

The methods of this disclosure enhance the economic viability and sustainability of downstream processing in biosynthesis of organic compounds. For this reason, several reuse and recycle processes may be included in the methods of this disclosure.

The methods of this disclosure conjugate several technologies that can be divided broadly into two steps: filtration and extraction. The processes of this disclosure combine these steps in a synergistic and successive way to obtain an isolated organic compound of high purity with energetic and economic efficiency.

The processes of this disclosure may be carried out on a continuous basis. In addition, the methods of this disclosure further admit of batch operations.

### Filtration Step

The beginning of the methods of this disclosure includes feeding a media containing an organic compound to a high-pressure reverse osmosis membrane. The media may be a reaction media in which the organic compound was synthesized. For example, the reaction media may be a fermentation media in which the organic compound may have been synthesized via microbial fermentation. Alternatively, or additionally, the media may be a reaction media in which the organic compound may have been synthesized in a cell-free process or a chemo-catalytic process. The media containing the organic compound is an aqueous media comprising cells and/or reaction components such as starting materials and/or other reaction products.

In the methods of this disclosure, the organic compound may be any one of alkanes, alkenes, alkynes, aromatic hydrocarbons, alcohols, phenols, ethers, aldehydes, ketones, carboxylic acids, esters, amides, acid anhydrides, amines, nitriles, alkyl halides, aryl halides, thiols, sulfides, sulfonic acids, phosphines, phosphates, and mixture or combinations thereof. Exemplary organic compounds useful in these methods include organic compounds synthesized in a biosynthesis process selected from the group consisting of acetic acid, propionic acid, butyric acid, valeric acid, caproic acid, lactic acid, and combinations thereof.

The aqueous media containing the organic compound is filtered through a high-pressure reverse osmosis (HPRO) membrane. In this HPRO filtration process, the aqueous media is pressurized and forced against a semi-permeable membrane. The solvent (water in the aqueous media) passes through the semi-permeable membrane, forming permeate on the other side, while solutes (such as salts, contaminants, impurities, cells, reaction components, and the organic compound) are retained on the feed side of the semi-permeable membrane and eventually expelled within a retentate containing the organic compound, which is then at a higher concentration in the retentate than in the aqueous media feed. This HPRO filtration process thus enables the selective separation of water from dissolved solutes, including the organic compound. The HPRO membrane may be a thin-film composite membrane comprising a polyamide active layer, which provides high salt rejection and water permeability, a polysulfone support layer, which adds structural stability, and a polyester backing, which serves as the foundation for the membrane structure.

In these methods, the HPRO membrane may operate at a pressure of at least 800 psi and the pressure may increase to 3000 psi (206 bar). The HPRO membrane may operate most efficiently in these methods at pressures between 800 psi and 1800 psi (55-124 bar). In these methods, the HPRO membrane may operate at a temperature between 5° C. and 45° C. (41-113° F.) and at a pH between 3 and 10.

In these methods, the HPRO filtration step achieves a concentration factor (CF; defined as the concentration of the organic compound in the HPRO retentate divided by the concentration of the organic compound in the) of at least 3.0, or at least 3.1, or at least 3.2, or at least 3.3, or at least 3.4, or at least 3.5, or at least 3.6, or at least 3.7, or at least 3.8, or at least 3.9, or at least 4.0, or between 3.5 and 5.0 for the organic compound. A lower CF may prevent scaling and fouling of the HPRO membrane.

During operation of the HRPO membrane, certain operational strategies may be employed to prevent fouling of the membrane. For example, an optional pre-filter (often called a polishing filter) may be employed upstream of the HPRO filter. This polishing filter may be one or more of a low-pressure multimedia filter, or a cartridge filter, or a microfiltration unit, or an ultrafiltration unit. These polishing filters can remove larger solids and particulates, such as protein aggregates and/or microbial cells, in the aqueous media prior to contact with the HPRO filter. Additionally, or alternatively, maintaining higher crossflow velocity of the media over the HPRO filter may reduce fouling (although higher crossflow rates may reduce CF). Additionally, or alternatively, periodic flushing of the HPRO membrane or chemical cleaning of the HPRO membrane can help remove fouling deposits during filtration.

The resulting retentate from the HPRO filtration step feeds the extraction procedures, which allow for the recovery of organic compounds in isolated or purified forms. Permeate, such as water, recovered from the filtration step, including any permeate from the optional polishing filter and/or permeate from the HPRO filtration, may be recycled to a bioreactor where the biosynthesis reaction that resulted in the creation of the organic compound originated. For example, permeate from the filtration step may contain water and cellular components from a fermentation broth and may be recycled to the bioreactor in which continued microbial fermentation produces additional organic compound.

### Extraction Step

In the methods of this disclosure, the organic compound is extracted next from the aqueous HPRO filtration retentate by one or more recovery processes. Exemplary recovery processes useful in extracting the organic compound from the retentate include liquid-liquid extraction, distillation, crystallization, electrodialysis, chromatography, adsorption, evaporation, and combinations of these processes.

### Liquid-Liquid Extraction

Liquid-liquid extraction (also known as solvent extraction and partitioning) is a method of separating compounds based on their relative solubilities in two different immiscible liquids, often water and an organic solvent. It is an extraction of a substance from one liquid phase into another liquid phase and is useful, for example, in the work-up after a chemical reaction to isolate and purify the product(s) or in removing valuable or hazardous components from waste or byproduct streams in a variety of industrial processes. The extracted substances may be organic such as fine chemicals, and liquid-liquid extraction therefore finds wide application including in the production of organic compounds, the processing of perfumes, nuclear reprocessing, ore processing, the production of petrochemicals, and the production of vegetable oils, among many other industries. Certain specific applications include the recovery of aromatics, recovery of homogeneous catalysts, manufacture of penicillin, recovery of uranium and plutonium, lubricating oil extraction, phenol removal from aqueous wastewater, and the extraction of acids from aqueous streams, the production of biofuels and organic acids derived from biomass fermentation that play a crucial role in sustainable chemical and energy industries.

In the methods of this disclosure, one liquid in the liquid-liquid extraction technique is the aqueous HPRO retentate containing the concentrated organic compound and the other liquid is an organic solvent. The organic compound dissolves into the organic solvent in which it is more soluble. The two immiscible liquids (the aqueous retentate and the organic solvent) are mixed to allow the organic compound to transfer into the organic solvent, and the two liquids are allowed to settle and are then physically separated. Using this technique, the organic compound is extracted from the aqueous HPRO retentate into the organic solvent, which can be further processed to purify it from the organic solvent. Liquid-liquid extraction can be conducted as a batch process (i.e., batch extraction; one-time mixing and separation), or continuous extraction (continuous transfer of the organic compound between the aqueous and organic phases.

Membrane-based liquid-liquid extraction (MBLLE) is a more advanced separation technique that combines traditional liquid-liquid extraction with membrane technology to enable continuous LLE with enhanced efficiency, selectivity, and process control. In the methods of this disclosure, MBLLE involves the intensive bulk mixing of the two immiscible liquid phases (the aqueous HPRO retentate and the organic solvent) to form emulsion. With one phase presents as droplets dispersed in the other phase, the effective contact area between the two phases is significantly increased, leading to accelerated selective transfer of the organic compound from the aqueous to the organic liquid. After the partition equilibrium is achieved, the MBLLE unit is then used to break the emulsion and separate it into pure aqueous and organic streams. The resulting organic solvent, now rich in the extracted compound, can be collected for further processing (i.e., isolation and/or purification of the organic compound). The membrane used for demulsification (i.e., breaking emulsions) in these MBLLE processes may be a hydrophobic membrane comprising a membrane material selected from polyethylene, high density polyethylene, perfluoroalkylate, polytetrafluoroethylene, ultra-high molecular weight polyethylene, a copolymer of ethylene and tetrafluoroethylene, polyphthalamide, polyamide, polyester, polysulfone, polyethersulfone, cellulose, cellulose acetate, regenerated cellulose, and combinations thereof. The pore size in the MBLLE membrane determines its efficiency for demulsification and phase separation. In the methods of this disclosure, the MBLLE membrane material preferably has a pore size of less than 2 μm, or less than 1 μm, or less than 0.5 μm. The flow across the membrane in the MBLLE process may be crossflow. In the MBLLE methods of this disclosure, the liquid-liquid extraction of the organic compound comprises extracting the organic compound into an organic phase with a solvent partition coefficient of at least 2.0 and/or with an extraction efficiency of at least 65%.

The mixing of the two liquids (aqueous HPRO retentate and the organic solvent) may be conducted by shaking or mixing the two liquids before introduction into the MBLLE apparatus. In the methods of this disclosure, this mixing is optionally conducted in a phase mixer that takes in the aqueous HPRO retentate and the organic solvent as separate input streams and thoroughly mixes the two immiscible liquids before the emulsion is directed into the MBLLE apparatus for demulsification.

In these methods, the HPRO retentate comprises the organic compound, and may also comprise components of the reaction media in which the organic compound was synthesized. Extracting the organic compound from the HPRO retentate thereby creates a spent HPRO retentate, which may be substantially depleted in the organic compound but may still contain elements of the reaction media. The methods of this disclosure may therefore optionally include recycling the spent HPRO retentate to a bioreactor for further reaction to produce more of the organic compound. For example, the spent HPRO retentate may contain elements of a fermentation media and this spent HPRO retentate may be recycled to a bioreactor for further (i.e., continuous) microbial fermentation that produces the organic compound. This recycling of the HPRO spent retentate may therefore continuously recycle reaction media to a bioreactor in a continuous process of biosynthetic production of the organic compound. Both HPRO and MBLLE techniques are non-thermal separation methods, and therefore, when they are used together in the methods of this disclosure, they may reduce energy consumption compared to distillation or evaporation.

### Distillation.

In the methods of this disclosure, the organic compound may be removed from the aqueous HPRO filtration retentate by a recovery process including distillation. Water from the aqueous HPRO filtration retentate (or solvent of another extraction method, such as MBLLE) can be distilled or flashed via distillation, leaving the organic compound behind. This approach will be most useful when the solvent comprising the organic compound comprises low molecular weight or high volatility solvents such as butanol, methyl isobutyl ketone, or triethylamine. It may be desirable to use relatively low-pressure conditions to facilitate the distillation. For example, processing at pressures on the order of about 500 mm Hg or below will be preferred. The use of a carrier gas and of pervaporation are also preferred. In some instances, the organic compound concentration which occurs during distillation may lead to the formation of condensation products. Distillation of the organic compound when the solvent containing the organic compound comprises a material of relatively low volatility may be performed at reduced pressure and elevated temperature conditions. For example, the distillation may be accomplished at pressures from about 0.2 to 100 mm Hg and at temperatures from about 80° C. to about 240° C. In some instances, the solvent phase containing the organic compound may contain materials of both high volatility and low volatility. When such is the case, multi-staged distillations may be performed to obtain isolation of the organic compound. Here again, a carrier gas and particularly pervaporation may be advantageous. Any organic solvent removed from the organic compound during distillation processes may be captured and optionally recycled to mixing with the HPRO retentate for further rounds of extraction. For example, as the organic compound is recovered and purified by distillation, a spent organic solvent may be captured from a distillation column and recycled to mix with the HPRO retentate before feeding to a liquid-liquid extraction process.

### Crystallization.

In the methods of this disclosure, the organic compound may be extracted from the aqueous HPRO filtration retentate by a recovery process including crystallization. Crystallization plays a crucial role in synthetic biology for isolating and recovering organic compounds with high purity and efficiency, but the process must be carefully controlled to optimize yield and maintain the desired physical properties of the organic compound. Crystallization of the organic compound proceeds via the formation of a solid crystal phase of the organic compound from the solution containing the organic compound. The process is driven by supersaturation, which occurs when the concentration of the organic compound exceeds its solubility limit. Crystallization of the organic compound as part of the extraction step in the methods of this disclosure may be particularly useful in separating the organic compound from other compounds or contaminants present in the solution containing the organic compound, while isolating the organic compound in a solid, stable, and often highly pure form. Isolation in this form may advantageously enable easier storage and handling of the organic compound. Crystallization of the organic compound may proceed by cooling (i.e., reducing the temperature of the solution decreases solubility, leading to crystal formation), solvent evaporation (i.e., slow removal of the solvent further increases the concentration of the organic compound, triggering crystallization), anti-solvent addition (i.e., adding a solvent in which the compound is less soluble causing precipitation of the organic compound), and/or chemical reactions (i.e., forming salts or derivatives that crystallize out). During these processes, careful control of the solution temperature, the mixing rate, and the solvent composition can control the speed and extent of the crystal growth and thus the purity of the recovered organic compound. Crystallization as part of the extraction of the organic compound from the HPRO retentate, especially with close control over these process parameters, may have the advantage of recovering the organic compound at a very high purity, and at minimal energy compared to, for example, chromatography. The purity of the organic compound extracted and recovered by crystallization may be at least 90%, or at least 95%, or at least 99%.

When the solvent containing the organic compound comprises non-polar solvent(s), such as toluene, crystallization may be a preferred process for extraction from the solvent. More specifically, organic compounds may crystallize readily from non-polar solvents.

### Electrodialysis.

Electrodialysis is an effective technique for isolating and recovering the organic compound created in synthetic biology processes from the HPRO retentate, particularly for organic compounds that are charged species appearing in complex biochemical mixtures, such as fermentation media. Electrodialysis operates using ion-exchange membranes and an applied electric field to selectively transport charged molecules, allowing for efficient purification and concentration of the targeted organic compounds. Electrodialysis employs alternating cation-exchange and anion-exchange membranes placed between electrodes such that when an electric potential is applied, cations migrate toward the cathode and anions migrate toward the anode. The use of selective membranes may allow only certain charged organic compounds to pass, creating zones of concentrated and depleted organic compounds. This can be a particularly useful extraction technique for organic acids, which exist as anions at neutral pH. These organic acids may be effectively separated from culture media while removing unwanted salts, significantly improving the purity of the organic acid. Electrodialysis may advantageously require less energy to isolate charged organic compounds from the HPRO retentate, compared to thermal-based separation methods such as distillation, particularly in methods where electrodialysis is combined with membrane filtration, chromatography, and/or liquid-liquid extraction.

### Chromatography.

Chromatographic separation, including reversed- and normal-phase high-performance liquid chromatography (HPLC), gas chromatography, affinity chromatography, and in particular, size-exclusion chromatography and ion-exchange chromatography, is useful in the isolation of organic compounds based on differential interactions of the organic compounds with a stationary phase and a mobile phase, allowing for the separation, purification, and recovery of specifically targeted organic molecules. In these methods, the solvent containing the organic compound (such as the HPRO retentate) is loaded into the chromatographic system, typically a chromatographic column. The organic compounds interact differently with the stationary phase in the chromatography system or column, leading to differential retention times such that the organic compounds elute from the column at different times based on their chemical properties. The collected fractions containing the organic compound may then undergo solvent removal (for example rotary evaporation) or crystallization to obtain the purified organic compound.

### Adsorption.

Another approach to isolation of the organic compound from the HRPO retentate containing the organic compound is through adsorption of the organic compound onto a solid adsorbent, followed by physical separation of the solid adsorbent from the liquid phase and eventual generation of the organic compound from the solid adsorbent. This method shows good efficiency when the solid phase has a high capacity for the organic phase, and an efficient regeneration cycle for long life of the solid phase within the organic compound extraction processes. The effectiveness of adsorption is isolating the organic compound depends upon the surface area of the adsorbent, the pore size and structure of the adsorbent material, chemical affinity between the adsorbent and the organic compound, and the operating conditions (pH, temperature, ionic strength) of the adsorption procedures. Adsorbent materials useful in the extraction methods of this disclosure include activated carbon (useful for isolation of hydrophobic organic compounds due to its high surface area), ion-exchange resins (effective for isolation of charged biomolecules such as amino acids, proteins, and organic acids), polymer adsorbents (useful for specific interactions tailored to individual organic compounds), silica-based adsorbent materials (useful for selective adsorption of polar compounds), and metal-organic frameworks (MOFs; useful for selective and high-capacity adsorption of organic compounds).

The desorption of the organic compound from the adsorbent may include techniques such as solvent elution (wherein organic solvents or aqueous buffers release the adsorbed organic compounds), pH or ionic strength changes (used to release adsorbed organic compounds from ion-exchange resins), temperature swings (heat can release adsorbed volatile organic compounds), and supercritical fluid extraction (CO₂ may be used for non-toxic recovery of the organic compound). The use of adsorption and subsequent desorption of the organic compound in the extraction methods of this disclosure has the advantages of being a highly selective separation process that may operate with high efficiency, while being a typically non-destructive purification, preserving bioactivity of organic compounds isolated using this technique. Adsorption may also be more environmentally friendly compared to the more energy-intensive distillation or solvent extraction processes. Fouling of the solid adsorbent and excessive dilution of the separated organic compound upon desorption, as well as difficulties with efficient process integration, may limit the applicability of adsorption extraction to fewer organic compounds within the methods of this disclosure.

### Evaporation.

Evaporation may be used in the isolation and recovery of the organic compound, particularly when separating volatile organic compounds, or concentrating samples containing the organic compound, or removing solvents from the HPRO filtrate—in particular, water. Organic compounds may be synthesized in aqueous or organic solvents and evaporation helps remove these solvents while retaining the organic compound. Selectively evaporating solvents or byproducts may form a more concentrated and purified organic compound. Evaporation includes the application of heat, and, depending on the solvent, may require high heat, such that controlled evaporation techniques are required to recover them without degradation, or evaporation may not be a technique available for organic compounds that are very sensitive to heat. Evaporative extraction techniques that may be used in the methods of the present disclosure include rotary evaporation (used for removing volatile organic solvents under reduced pressure, which may also be applicable for extraction of organic compounds sensitive to heat), vacuum evaporation (which utilizes a vacuum chamber to reduce pressure and enhance evaporation at lower temperatures and may make the evaporation step faster; also useful for recovering organic compounds that are thermally unstable), spray drying (which converts liquid samples into dry powder using rapid solvent evaporation (which is particularly useful in the isolation of enzymes and proteins), thin-film evaporation (which uses a thin liquid film under reduced pressure to rapidly evaporate solvents), freeze-drying (or “lyophilization” which is a sublimation process where the solvent, typically water, is frozen and removed under vacuum). The efficiency of these evaporation in the extraction step of the methods of this disclosure may depend on the boiling point differences between the solvent and organic compound (a greater boiling point difference will improve separation), and the vacuum level that may be applied during evaporation of the solvent (in particular, the application of vacuum can reduce the effective boiling point of the solvent, enabling efficient solvent evaporation at lower temperatures). Additionally, evaporation processes may enable the recovery (for example through condensation or distillation) of solvents, such as ethanol, methanol, acetone, which can be recycled within the recovery methods of this disclosure to minimize waste and cost.

Systems of this Disclosure

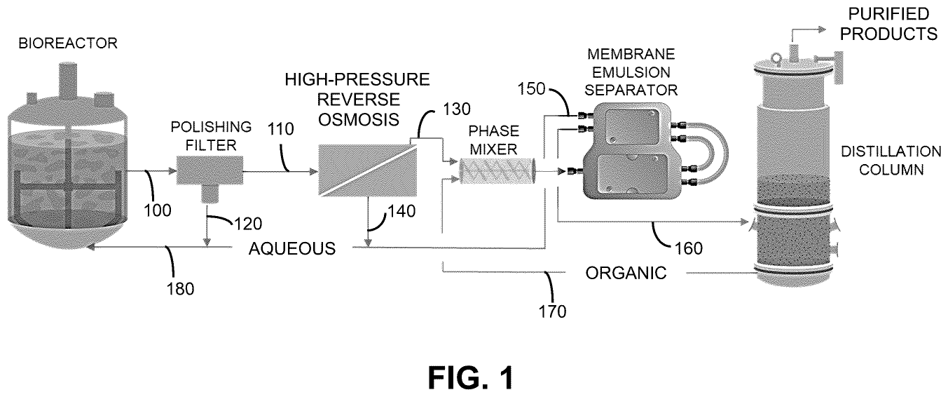

This disclosure also provides systems for extracting an organic compound from a media, such as a cell free reaction media or a fermentation broth. FIG. 1 illustrates one exemplary embodiment of these systems of this disclosure. It should be made clear to those skilled in the art, however, that modifications and variations are possible from the embodiment illustrated in FIG. 1 without deviating from the scope of the present disclosure.

FIG. 1 corresponds to a system flowchart wherein unit operations are named and materials stream flows are represented by the numerals 100 to 170.

The basic sequence of unit operations in these systems of this disclosure, including major streams, is depicted in FIG. 1 in addition to additional and optional processing units and material flows. The unit operations of these systems include a high-pressure reverse osmosis (HPRO) unit configured to accept an aqueous stream comprising an organic compound from a bioreactor and concentrate the organic compound in an aqueous HPRO filtration retentate and an extraction unit, fluidly connected to the HPRO unit, configured to isolate the organic compound from the aqueous HPRO filtration retentate to form an isolated organic compound and a spent aqueous HPRO filtration retentate.

Referring to FIG. 1, the HPRO unit accepts an aqueous stream (100) comprising an organic compound from the bioreactor. The system embodiment depicted in FIG. 1 includes an optional polishing filter that may remove large particulate or reaction products, such as microbial cells from a fermentation media or protein aggregates from a biosynthesis reaction media. Such large particulate or reaction products removed from the aqueous stream (100) may be returned (120) to an aqueous return feed (180) to the bioreactor. In this configuration, aqueous stream (100) comprising an organic compound from the bioreactor is polished of large particulate before entering the HPRO unit.

The aqueous stream (100) comprising an organic compound is concentrated in the HRPO unit as solvent (i.e. water) permeates through the HPRO membrane. The HPRO unit comprises an HPRO membrane that is preferably operated at a pressure between 800 psi and 1800 psi. The HPRO retentate (130) is an aqueous stream comprising the organic compound in a higher concentration than the concentration of the organic compound in the aqueous stream (100) from the bioreactor. The permeate from the HPRO unit is an aqueous stream (140) that may be returned to an aqueous return feed (180) to the bioreactor.

The aqueous HPRO retentate stream comprising the organic compound is directed to the extraction unit. As depicted in the embodiment illustrated in FIG. 1, the extraction unit is composed of a membrane emulsion separator, specifically a membrane-based liquid-liquid extraction (MBLLE) unit, in fluid communication with, and upstream of, a distillation column. Together, the MBLLE separator and the distillation column comprise the extraction unit of the system depicted in the embodiment shown in FIG. 1. Within this embodiment shown in FIG. 1, an optional phase mixer is included, which receives the aqueous HPRO retentate stream (130) comprising the organic compound as well as an organic solvent stream (170) and mixes these two immiscible liquids to form an emulsion that is directed into the membrane emulsion separator.

In the MBLLE separator, the organic compound partitions into the organic solvent across a membrane to form a solvent comprising the organic compound and a spent aqueous HPRO retentate. The spent aqueous HPRO retentate (150) exits the MBLLE separator and may be returned to an aqueous return feed (180) to the bioreactor. The solvent comprising the organic compound (160) exits the MBLLE separator and is directed into the distillation column. The organic compound is isolated from the organic solvent by distillation and the purified products (i.e., purified organic compound) is recovered from the distillation column. The organic solvent, stripped of the organic compounds in the distillation column (170), may be returned to be mixed with aqueous HPRO retentate stream exiting the HPRO unit.

While the extraction unit depicted in the exemplary system embodiment illustrated in FIG. 1 is a membrane emulsion separator in fluid communication with a distillation column, it is to be understood that within the systems of this disclosure, the extraction unit comprises a recovery process selected from the group consisting of liquid-liquid extraction, distillation, crystallization, electrodialysis, chromatography, adsorption, evaporation, and any combination thereof.

In these systems, the organic compound may include any alkanes, alkenes, alkynes, aromatic hydrocarbons, alcohols, phenols, ethers, aldehydes, ketones, carboxylic acids, esters, amides, acid anhydrides, amines, nitriles, alkyl halides, aryl halides, thiols, sulfides, sulfonic acids, phosphines, phosphates, and any combinations thereof.

### EXAMPLES

### Example 1: Carboxylic Acid Concentration in Downstream Bioprocessing Using High-Pressure Reverse Osmosis

During the production of many bio-based chemicals from fermentation and enzymatic processes, product separations frequently represent the most expensive and energy-intensive unit operations in an integrated process, often due to the low concentrations of target bioproducts. In this example, a high-pressure reverse osmosis (HPRO) was utilized to concentrate an exemplary organic compound, butyric acid, produced by a fermentation biosynthesis, prior to downstream extraction. Through both modeling and experimental measurements, major factors were identified that can limit the maximum achievable concentration factor compared with conventional reverse osmosis (RO) membranes.

Membranes are of interest for product concentration applications due to their high selectivity and flexibility, low process energy requirements and chemical usage, and broad applications. To that end, as described herein, the feasibility of using membranes to recover and concentrate acids from fermentation broths has been assessed for the recovery of carboxylic acids, dicarboxylic acids, and alcohols, with many of these membrane concentration processes often reporting bioproduct concentration factors (CF) from fermentation broth ranging from 1.7-2.8. For example, it was reported that a reverse osmosis (RO) membrane successfully concentrated a mixture of carboxylic acids by a CF of 2.8 (J. M. Domingos, et al., Separation and Purification Technology, 2022, 290:120840). In other studies, a maximum CF of 1.7 was achieved by RO-membrane dewatering of a model butyric acid solution, and separately, a commercial nanofiltration (NF) membrane concentrated 1,3-propanediol from an ultrafiltered fermentation broth with a CF of 2.5 (Y. H. Cho, et al., Industrial & Engineering Chemistry Research, 2012, 51:10207-19).

It is expected that a higher operating pressure in membrane-driven concentration steps could enable a higher product CF, indicated by the observed enhanced membrane separation performance in terms of both membrane permeate flux and rejection of biomolecules from broth. However, most modern NF and RO membranes are thin film composite membranes constructed from polyamide coating onto a polysulfone support layer, and these systems are often limited by a maximum operating pressure of 41 and 69 bar, respectively. Higher operating pressures would lead to >50% membrane performance loss due to support layer compaction and densification.

Therefore, as reported herein the potential of HPRO membranes in bioprocessing was evaluated by integrating a membrane bioproduct concentration step into a continuous ISPR process that aims to separate and purify butyric acid from fermentation broth. This case study was used to evaluate the technical feasibility and economic and environmental impacts of the HPRO-integrated ISPR process compared to conventional and RO-integrated processes. An objective of the work described herein was to understand the major limiting factors of the membrane product concentration process capability that hinder the achievement of a higher product CF. HPRO membrane throughput, butyric acid CF, and product recovery were then systematically compared with conventional RO membranes, and their impact on the efficiencies of the subsequent unit operations, LLE and distillation. Using process modeling, this example demonstrates that HPRO-integrated ISPR exhibits significantly improved economics and reduced greenhouse gas (GHG) emissions.

### A. Membrane-Based Product Concentration Modeling.

A model of membrane-based product concentration that is valid for both batch and continuous operations was built. The model was used to assess the impacts of membrane selection and operating conditions on the maximum product concentration factors (CF), defined as the bioproduct concentration in the concentrated stream of the membrane retentate divided by the bioproduct concentration in the initial feed stream to the membrane. This model is intended to generally represent nonporous membrane (NF/RO/HPRO) product concentration. By definition, a higher product CF can be achieved if more solvent is removed as the membrane permeate, while most of the bioproduct (if not all) is retained by the membrane as retentate. Consequently, it was hypothesized that membrane intrinsic properties such as membrane hydraulic resistance and solute permeability coefficient would affect membrane product concentration performance. Notably, for most bioproduct concentration processes from synthesis processes, such as fermentation broth, under a constant applied pressure, there is a maximum permeate volume recovery as the membrane permeate flux continues to decline until it reaches zero. The flux decline over the course of the membrane concentration is expected as the concentrated feed solution osmotic pressure continues to increase, and thus leads to a decreased transmembrane pressure gradient, which is the solvent permeation driving force for NF/RO/HPRO membranes.

In addition, the increased viscosity of the concentrated feed solution from the biosynthesis process and membrane fouling can also contribute, at least partially, to the membrane permeate flux decline. As membranes may foul during operation, and the fouling layer serves as an extra resistance layer to solvent transportation, membrane permeate flux can be expressed as a function of membrane fouling. Assuming the applied pressure is constant, it is expected that the decline in membrane permeate flux over the course of product concentration will limit the maximum product CF, and that this can be attributed to the combined effect of increased solution osmotic pressure and viscosity due to product concentration, as well as membrane fouling. As increasing solution osmotic pressure and viscosity are inevitable for product concentration from a given feed solution, to maximize product CF, it is thus critical to have the membrane process operated under a higher applied pressure (that membrane can withstand), and select a membrane and/or membrane operating conditions that would lead to the lowest membrane fouling. Assuming the pressure applied to the membrane is constant, it is expected that the decline in membrane permeate flux over the course of bioproduct concentration will limit the maximum bioproduct CF, and that this can be attributed to the combined effect of increased solution osmotic pressure and viscosity due to bioproduct concentration, as well as membrane fouling. As increasing solution osmotic pressure and viscosity are inevitable for bioproduct concentration from a given feed solution, to maximize product CF it is thus critical to have the membrane process operated under a higher applied pressure (that the membrane can withstand), and select a membrane and/or membrane operating conditions that would lead to the lowest membrane fouling propensity.

To simplify the model, it was assumed that mass transfer and solute permeability are constant throughout the membrane concentration process assuming the membrane surface shear rate is constant (controlled by membrane crossflow velocity in a continuous cross flow filtration setup). Membrane selectivity of the targeted bioproducts as well as other impurities in the feed solution (i.e., inorganic salts) are assumed to be the same, and solution osmotic pressure and viscosity increase linearly with product CF in membrane concentrate. Fouling resistance is also assumed to have a constant accumulation rate on membrane surface throughout the course of membrane product concentration process until it reaches the maximum achievable product CF.

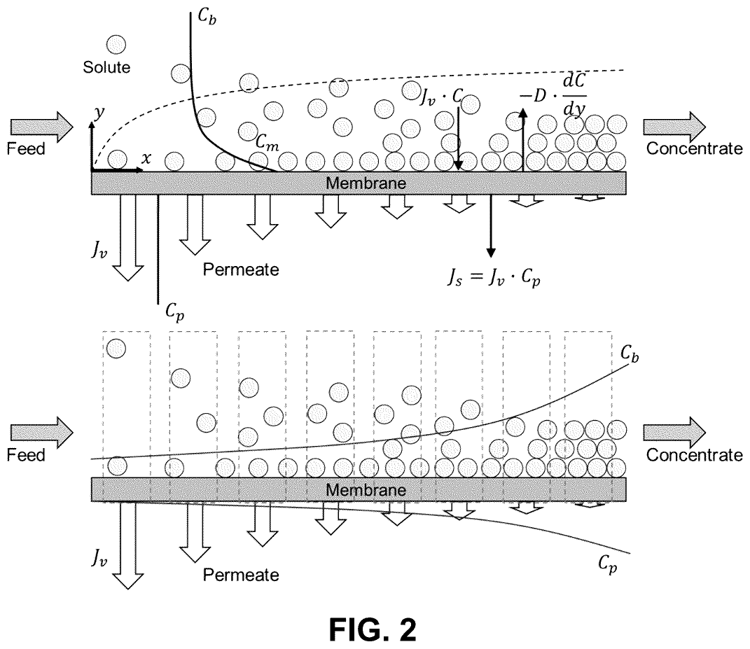

The developed model is also valid for continuous membrane product concentration processes, as it can be viewed as equivalent to infinite stages of a dead-end membrane product concentration (FIG. 2). Due to the concentration polarization phenomenon developed both vertically (y in FIG. 2), as indicated by the higher solute concentration at the proximity to membrane surface compared to the bulk solution, and horizontally (x in FIG. 2), as indicated by the higher solute concentration in the bulk solution at membrane exit compared to the inlet, the membrane permeate flux continues to decrease along the cross-flow direction while the membrane permeate solute concentration also continues to buildup. The decreased membrane permeate flux and increased solute permeate concentration are both expected in the dead-end and continuous cross-flow membrane product concentration processes, but as a function of time and as a function of membrane position along the cross-flow direction, respectively. Similar to the dead-end membrane product concentration process, the maximum product CF under a given applied pressure for the continuous cross-flow membrane product concentration process can be achieved when the membrane permeate flux at the membrane exit reaches zero. To use the above derived model to represent a continuous cross-flow membrane product concentration process, all the volume parameters need to be replaced with volumetric flow rates, and the derivatives taken with time need to be replaced with membrane position.

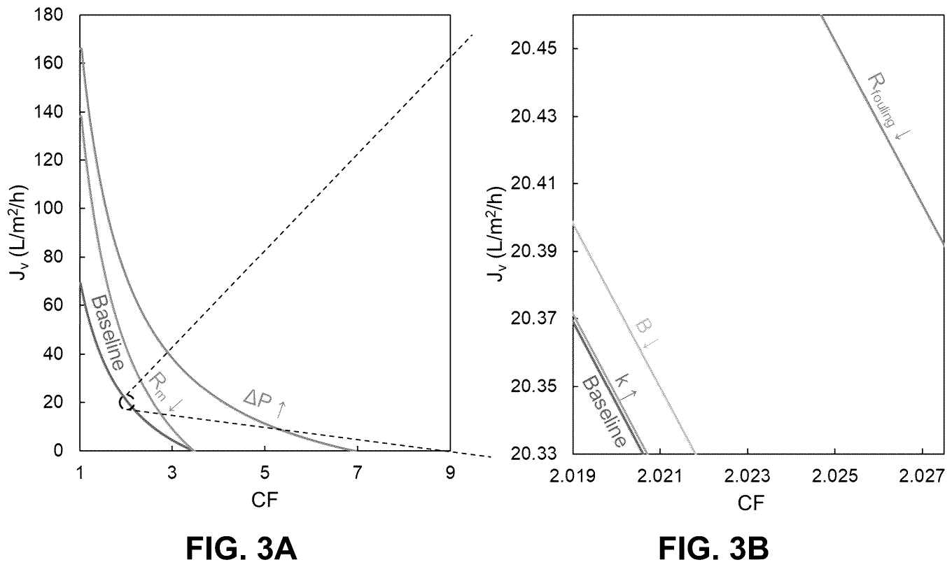

The model identified factors that may affect product CF during product concentration utilizing HPRO membranes: 1) the initial feed solution conditions in terms of osmotic pressure and solution viscosity; 2) intrinsic membrane properties in terms of membrane hydraulic resistance and membrane solute permeability coefficient; 3) membrane operating conditions and the resulting membrane performance in terms of applied pressure, membrane solute mass transfer coefficient, and fouling resistance. From the model, the mass transfer coefficient, together with membrane hydraulic resistance, can be used to determine membrane surface concentration polarization. For a given feed solution, such as a fermentation broth, the initial osmotic pressure and viscosity are set. Thus, one can only manipulate the other factors to tune membrane product concentration performance via membrane selection and operating condition optimization. To visualize the model and assess the impact of each factor on product CF, values were selected for the required variables in a baseline scenario and then changed one variable at a time to understand the impact of each on membrane performance (Table 1, FIG. 3).

TABLE 1
Variable set values for model visualization.

| | ΔP | Rₘ | R_{fouling} | B (L · | k (L · |
|---|---|---|---|---|---|
| Variables | (psi) | (m⁻¹) | (m⁻¹) | m⁻² · h⁻¹) | m⁻² · h⁻¹) |
| Base | 800 | 1.3 × 10¹⁴ | 0 | 3.9 | 137 |
| Control | 1,600 | 6.4 × 10¹³ | 1.3 × 10¹⁴ | 2 | 300 |

Surprisingly, according to the derived model, from a stream with a given initial solution osmotic pressure and viscosity, such as a fermentation broth, the feed applied pressure is the only factor identified that affects the theoretical maximum CF for membrane product concentration. In a batch process model, membrane hydraulic resistance would affect how fast the membrane concentration process can be completed, but not the maximum achievable product CF. In other words, a membrane with a higher permeability coefficient or smaller hydraulic resistance can reach the maximum CF in a shorter time, while the absolute value of maximum CF remains the same. Additionally, in a continuous process, a membrane with a higher permeability coefficient can reach the maximum CF via a smaller membrane area. Similar to membrane hydraulic resistance, the membrane solute mass transfer, solute permeability coefficient, and membrane fouling do not affect the absolute value of maximum CF but can accelerate the rate of membrane product concentration for a batch process and reduce the required membrane area for a continuous process, although less significantly. The results from the model indicate that HPRO membranes could be promising for bioproduct concentration. Unlike the conventional RO membranes, whose maximum operating pressure lies in the range of 800-1,000 psi (55-69 bar), HPRO membranes can endure much higher operating pressures of up to 1,740 psi (120 bar; according to the manufacturer's specification sheet) and could, therefore, enable enhanced product concentration capacity.

### B. Experimental Measurement of Membrane-Based Product Concentration.

Experiments were conducted to collect and validate the membrane bioproduct concentration model described above. To confirm the prediction that only minimum impacts of membrane permeability and selectivity have on the maximum CF achievable for bioproduct concentration, the perm-selective performance of various commercial membranes were evaluated, including 21 polyamide RO membranes (Dow XLE, XFR, BW30, SW30HRLE, SEAMAXX, CR100, XUS1203, Toray UTC-82V, UTC-73AC, UTC-73HA, UTC-73UAC, TriSep SB50, X201, ACM1, ACM2, ACM3, ACM4, Suez (GE) AK, SE, and AG) and one polyamide HPRO membrane (XUS180808, DuPont Water Solutions; a spiral-wound element with polyamide thin-film composite membrane having 120 bar (1,740 psi), ultra-high feed pressure capability, and Maximum Element Pressure Drop of 15 psig (1.0 bar)). A short-chain carboxylic acid, butyric acid, produced via fermentation with Clostridium tyrobutyricum, was used as a case study to evaluate the product concentration capacity of the commercial RO and HPRO membranes. Membrane performance was evaluated using a 300 mL dead-end stirred RO cell (HP4750X, Sterlitech Corporation), which can accommodate a flat sheet RO or HPRO membrane coupon with an active membrane area of 14.6 cm². Transmembrane pressure was supplied with compressed N2 (99.5% purity), and the permeate volumetric flow rate was monitored with an in-line liquid flow meter (SLS-1500, Sensirion AG). Prior to any membrane test, all membrane coupons were immersed in D.I. water overnight. The immersed membranes were first compacted with D.I. water under pressure 55.2 bar (approximately 800 psi) for RO and 110.3 bar (approximately 1,600 psi) for HPRO at a stir rate of 700 rpm at 20° C. for 3 hours until the permeate flux stabilized. The membrane permeate flow rate was then measured over a transmembrane pressure range of 27.6-55.2 bar (400-800 psi) for RO and 55.2-110.3 bar (800-1,600 psi) for HPRO, and the membrane water permeability coefficient was determined. It is noted that D.I. water has osmotic pressure of zero. The membrane selectivity of butyric acid, in terms of observed rejection and intrinsic rejection and solute permeability coefficient, was determined with a model feed solution (i.e., 15 g/L butyric acid in D.I. water with pH adjusted to 5 using 1 N NaOH solution). The model feed solution represents a single component model solution to mimic the C. tyrobutyricum fermentation broth. The target butyric acid titer of 15.0 g/L in the bioreactor at pH 5 was chosen to minimize product inhibition for the bacterium while creating an acceptable driving force for organic solvent extraction. At each transmembrane pressure, 1 mL of a permeate sample was collected for high-performance liquid chromatography (HPLC) analysis after the permeate flux stabilized for approximately 10 min. The operating conditions for the RO and HPRO membrane separation testing were:
Upstream clarifying/polishing filter: 1 kDa regenerated cellulose UF membrane (with an active area of 13.4 cm²) using a 50 mL dead-end stirred UF cell at 3.4 bar and stir rate of 400 rpm
Membrane material for all membranes tested: polyamide
Membrane permeate flow rate was then measured over a
Transmembrane pressure range for RO membranes: 27.6-55.2 bar (400-800 psi)
Transmembrane pressure range for HPRO membranes: 55.2-110.3 bar (800-1,600 psi)
Model feed solution: 15 g/L butyric acid in D.I. water
pH of feed solution: adjusted to pH 5 using 1 N NaOH
Stir rate of feed solution: 700 rpm.
Operating temperature: 20° C.
Range of water permeability coefficient: 1.0-7.5 L/m²/h/bar
Range of membrane selectivity (A/B) for butyric acid (as a function of water permeability coefficient): 0.1-1.8 bar-1
Range of decrease in permeate flux at end of concentration tests (all membranes): 93.2-97.7%
Permeate flow rate at termination of concentration tests: 0.1 mL/min
Time to complete concentration of 100 mL clarified fermentation broth (RO membranes): 140-200 min
Range of butyric acid CF (RO membranes): 2.6-3.2
Time to complete concentration of 100 mL clarified fermentation broth (HPRO membranes): 300 min
Range of butyric acid CF (HPRO membranes): 2.6-3.2

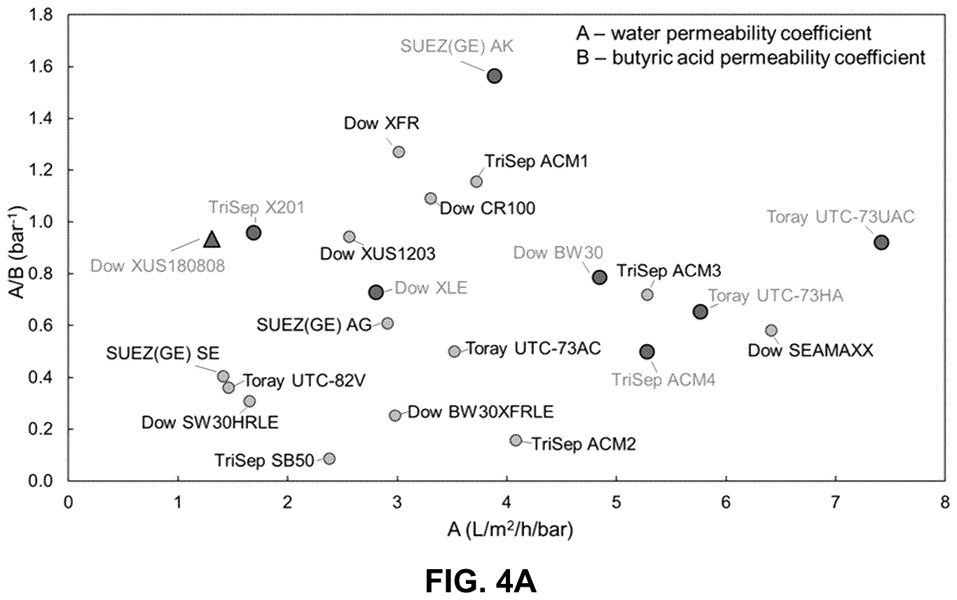

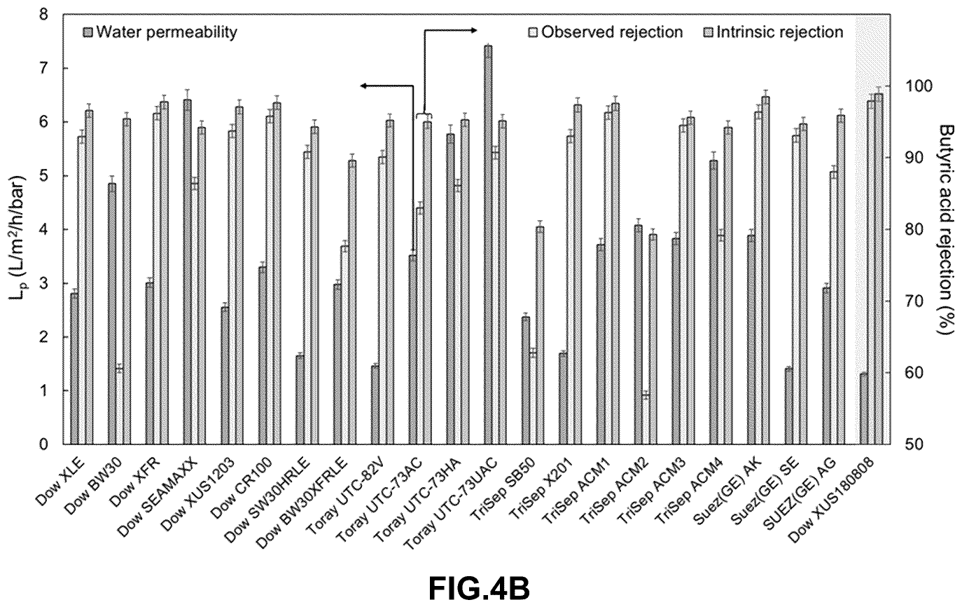

This experimental performance evaluation indicated that commercial polyamide RO and HPRO membranes demonstrated a wide range of performance in terms of water permeability and butyric acid selectivity. For example, the tested polyamide RO and HPRO membranes' water permeability coefficients range from 1.3 to 7.4 L·m⁻²·h⁻¹·bar⁻¹, butyric acid permeability coefficients are from 1.4 to 27.3 L·m⁻²·h⁻¹, and observed and intrinsic butyric acid rejections span 56.9% to 97.9% and 79.3% to 98.9%, respectively (FIG. 4, Table 2). In Table 2, note that membrane perm-selectivity is presented as a ratio of its water and solute permeability coefficients (A/B), where water permeability coefficient (Lp) is denoted as A. (Dow XUS180808 is the commercial HPRO with maximum operating pressure of 120 bar (1740 psi), while the rest of commercial RO membranes have maximum operating pressure of 68.9 bar (1000 psi)). Membrane butyric acid selectivity was determined with a model feed solution (i.e., 15 g/L butyric acid in D.I. water with pH adjusted to 5 using 1 N NaOH solution) at stir rate of 700 rpm, temperature of 20° C., and at a transmembrane pressure range of 27.6-55.2 bar (400-800 psi) for RO and 55.2-110.3 bar (800-1,600 psi) for the HPRO membrane. The data points with larger and darker marks in FIG. 4A represent the membranes selected for the subsequent butyric acid concentration tests with C. tyrobutyricum fermentation broth.

TABLE 2
Summary of perm-selective properties of the commercial RO and HPRO membranes.

| Membrane | Membrane | | | | | |
|---|---|---|---|---|---|---|
| Type | Name | Lₚ (L · m⁻² · h⁻¹ · bar⁻¹) | Rₒ (%) | Rᵢ (%) | B (L · m⁻² · h⁻¹) | A/B (bar⁻¹) |
| RO | Dow ™ XLE | 2.81 | 92.90 | 96.56 | 3.87 | 0.73 |
| RO | Dow ™ BW30 | 4.85 | 60.56 | 95.44 | 6.18 | 0.78 |
| RO | Dow ™ XFR | 3.01 | 96.22 | 97.85 | 2.37 | 1.27 |
| RO | Dow ™ CR100 | 3.30 | 95.76 | 97.74 | 3.02 | 1.09 |
| RO | Dow ™ SW30HRLE | 1.65 | 90.78 | 94.29 | 5.31 | 0.31 |
| RO | Dow ™ BW30XFRLE | 2.98 | 77.70 | 89.59 | 11.72 | 0.25 |
| RO | Dow ™ SEAMAXX | 6.41 | 86.43 | 94.16 | 11.00 | 0.58 |
| RO | Dow ™ XUS1203 | 2.56 | 93.70 | 97.09 | 2.71 | 0.94 |
| RO | Toray ™ UTC-82V | 1.46 | 90.09 | 95.20 | 4.03 | 0.36 |
| RO | Toray ™ UTC-73AC | 3.52 | 83.00 | 95.03 | 7.03 | 0.50 |
| RO | Toray ™ UTC-73HA | 5.77 | 86.11 | 95.26 | 8.85 | 0.65 |
| RO | Toray ™ UTC-73UAC | 7.42 | 90.71 | 95.13 | 8.06 | 0.92 |
| RO | TriSep ™ X201 | 1.69 | 92.99 | 97.37 | 1.76 | 0.96 |
| RO | TriSep ™ ACM1 | 3.72 | 96.34 | 97.55 | 3.22 | 1.16 |
| RO | TriSep ™ ACM2 | 4.08 | 56.88 | 79.34 | 25.90 | 0.16 |
| RO | TriSep ™ ACM3 | 3.83 | 94.49 | 95.64 | 5.32 | 0.72 |
| RO | TriSep ™ ACM4 | 5.28 | 78.75 | 93.96 | 10.60 | 0.50 |
| RO | TriSep ™ SB50 | 2.38 | 62.83 | 80.40 | 27.34 | 0.09 |
| RO | SUEZ ™ (GE) AG | 2.91 | 88.04 | 95.90 | 4.77 | 0.61 |
| RO | SUEZ ™ (GE) AK | 3.89 | 96.43 | 98.46 | 2.49 | 1.56 |
| RO | SUEZ ™ (GE) SE | 1.41 | 93.66 | 95.33 | 3.50 | 0.40 |
| HPRO | Dow ™ XUS180808 | 1.31 | 97.89 | 98.85 | 1.40 | 0.94 |

Notably, among the tested membranes, the HPRO membrane exhibited the best butyric acid selectivity in terms of the highest butyric acid rejections and the lowest butyric acid permeability coefficient, but at the cost of the lowest water permeability coefficient (FIG. 4A, Table 2). This is expected, as to withstand such a high operating pressure range, the HPRO membrane must have a sufficiently dense support layer (i.e., a partial sponge-like structure) and a relatively large selective layer thickness (≥250-300 nm) to ensure its integrity and structural robustness, which leads to low membrane water permeability and high membrane selectivity. Membrane perm-selective properties are also illustrated in FIG. 4A, depicting the conventional plot of membrane water/solute permeability coefficients ratio (A/B) as a function of membrane water permeability coefficient (A). To separate butyric acid from water, it is desirable to select a membrane with both a high water permeability coefficient, indicating high membrane throughput, and high water/solute permeability coefficients ratio, indicating high membrane solute (butyric acid) selectivity. For butyric acid concentration, according to the model, it is expected that a membrane with higher water permeability coefficient could enable an accelerated butyric acid concentration process in batch operation and a reduced membrane area requirement in continuous operation, while the absolute values of maximum butyric acid CF are predicted to be similar for all tested RO membranes under the same applied pressure.

To validate the model prediction that applied pressure is the only factor affecting membrane maximum CF during product concentration, seven commercial RO membranes and one HPRO membrane with different perm-selective properties and maximum operating pressures were selected to concentrate butyric acid from C. tyrobutyricum fermentation broth (i.e., 15 g/L butyric acid in D.I. water with pH adjusted to 5 using 1 N NaOH solution; elemental composition of the C. tyrobutyricum fermentation broth: B 5.4 ppm; Ca 16 ppm; Fe 4 ppm; K 1743 ppm; Mg 45 ppm, Mn 5.7 ppm; Na 3675 ppm; Si 31 ppm; P 833.8 ppm; S 816 ppm. The selected RO and HPRO membranes were Dow XLE, Dow BW30, Toray UTC 73HA, Toray UTC-73UAC, TriSep X201, TriSep ACM4, Suez (GE) AK, and Dow XUS180808 membranes with water permeability range of 1.3-7.4 L·m⁻²·h⁻¹·bar⁻¹ and butyric acid permeability range of 1.4-10.6 L·m⁻²·h⁻¹. Before the RO concentration process, the C. tyrobutyricum fermentation broth was clarified via a 1 kDa regenerated cellulose UF membrane (with an active area of 13.4 cm²) using a 50 mL dead-end stirred UF cell (Amicon 8050, Millipore Corporation) at 3.4 bar (49 psi) and stir rate of 400 rpm, with the intention to mimic a continuous crossflow cell retention unit operation in an ISPR process. The clarification step removed the solid fraction of the fermentation broth (i.e., cells and cell debris) and reduced the presence of soluble small organic molecules (e.g., protein). The UF permeate was collected and sent to the membrane concentration process as the feed solution, to lower RO and HPRO membrane fouling propensity during butyric acid concentration. During the fermentation broth clarification step, negligible UF membrane fouling was observed, as indicated by the permeate flux remaining constant with up to 80% total permeate recovery. Similar to the experiments described above, the selected RO and HPRO membranes, after immersion in D.I. water overnight (>24 h), were compacted with D.I. water under a transmembrane pressure of 55.2 bar (approximately 800 psi) and 110.3 bar (1,600 psi), respectively, at stir rate of 700 rpm at 20° C. for 3 h allowing the permeate flux to stabilize. The compacted RO and HPRO membranes were then used to concentrate 100 mL of clarified C. tyrobutyricum fermentation broth using the dead-end stirred RO cell. Membrane concentration tests were carried out at stir rate of 700 rpm at 20° C., with the membrane permeate flow rate monitored with an in-line liquid flow meter (SLS-1500, Sensirion AG), and permeate samples collected over time. The membrane permeate flux was expected to decrease throughout the concentration tests, which were terminated when the membrane permeate flow rate fell below 0.1 mL/min, with samples of the final concentrate collected. Solution pH and conductivity were measured using pH (Mettler Toledo™ FiveEasy™ F20) and conductivity (Mettler Toledo SevenMulti S47) meters for clarified fermentation broth and collected permeate and concentrate samples from each membrane concentration test. The above liquid samples were also collected for HPLC analysis to calculate product CF and product recovery (PR).

According to the model described above, for a given membrane, the reduced membrane permeate flux during product concentration is a combined effect of membrane fouling and increasingly concentrated fermentation broth osmotic pressure and viscosity. To evaluate the contribution of membrane fouling to the membrane permeate flux decline, each individual contribution to the overall flow resistance was determined. Membrane hydraulic resistance of the pristine membrane was determined with D.I. water using the Darcy's law. Membrane product concentration testing from fermentation broth with constant applied pressure was terminated after the membrane permeate flow rate fell below 0.1 mL/min. After the membrane product concentration test was terminated, membrane samples were flushed with D.I. water for two minutes to remove the product residue remaining on the membrane surface, and the flushed membrane permeate flux in D.I. water was calculated to determine the fouling resistance. The contribution of membrane fouling to the decline in membrane permeate flux was then calculated.

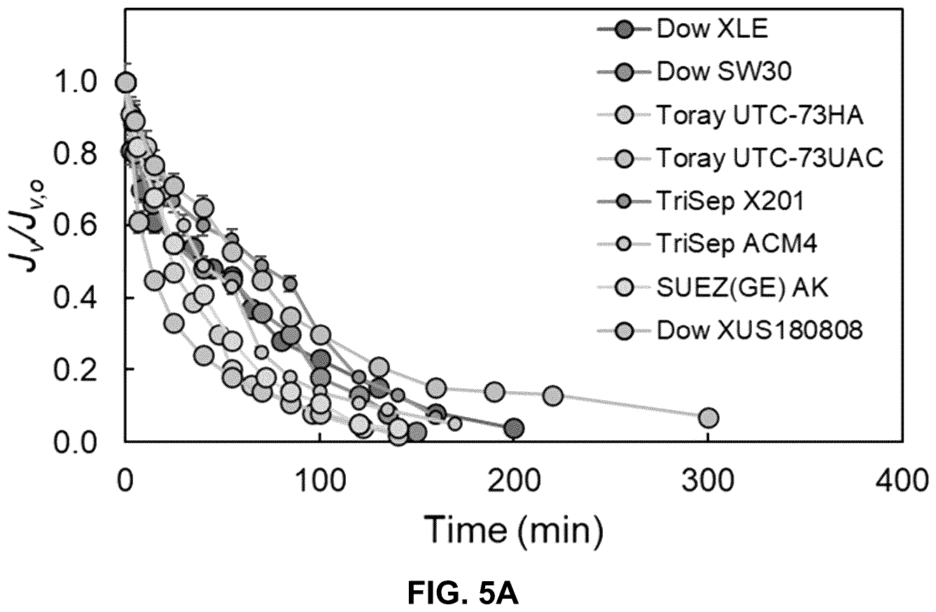

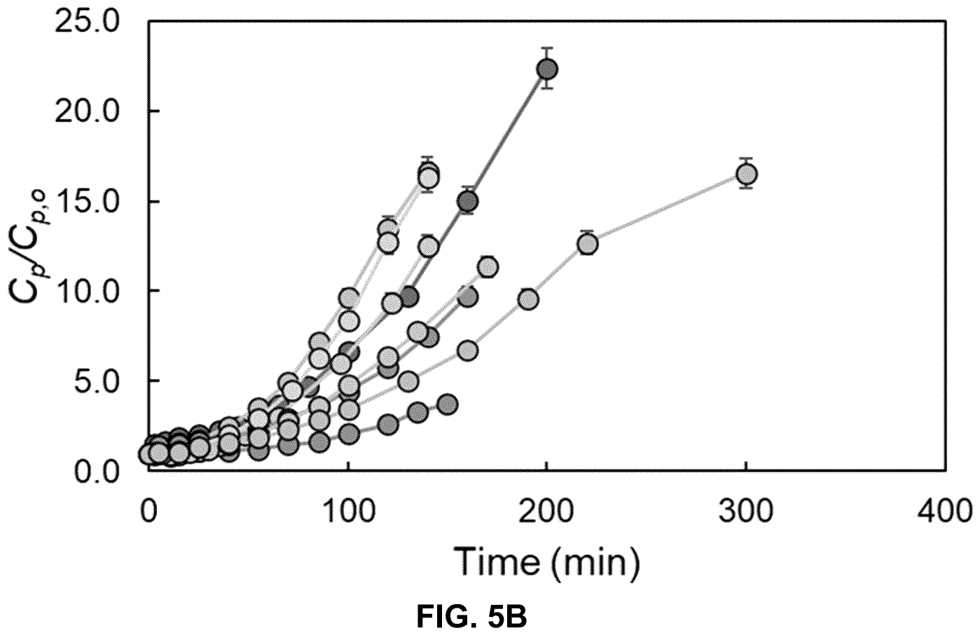

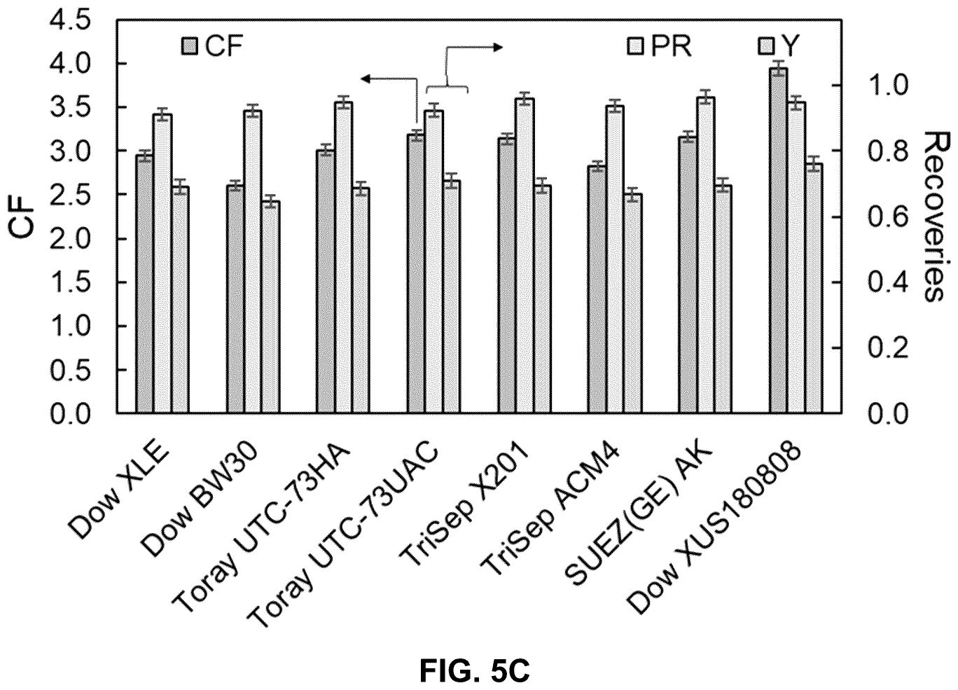

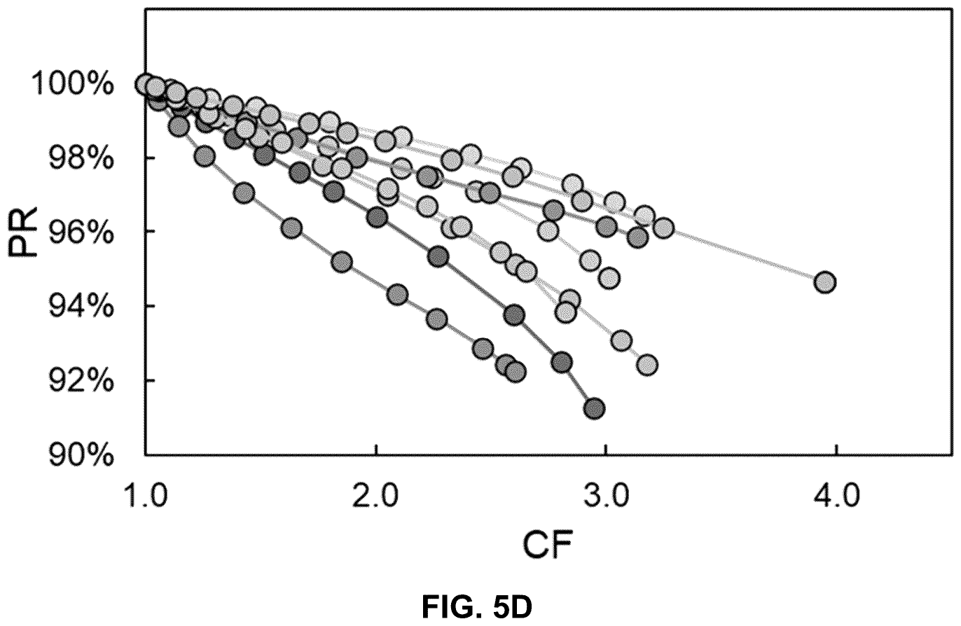

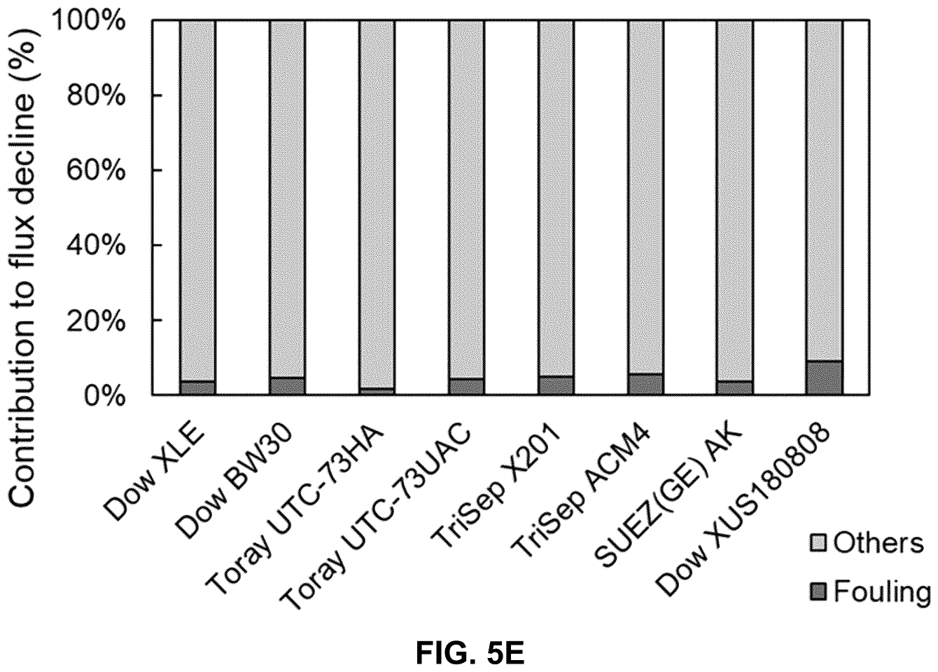

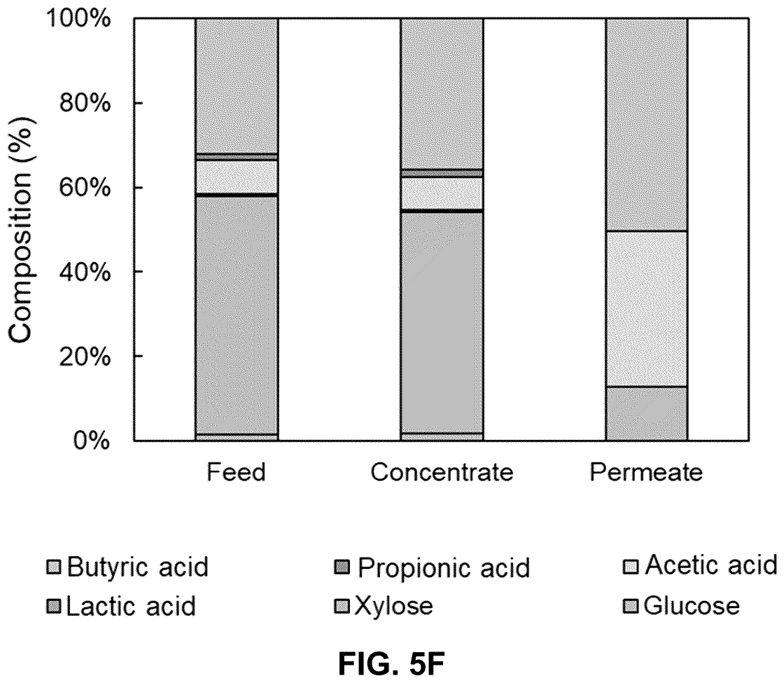

At the end of the clarified fermentation broth concentration tests, all eight tested membranes demonstrated 93.2-97.7% decreased permeate flux and increased permeate butyric acid concentration by a factor of 3.7-22.4 (FIGS. 5A and 5B). After termination (when the permeate flow rate fell below 0.1 mL/min), all seven RO membranes completed their concentration of 100 mL clarified fermentation broth within 140-200 minutes, and achieved a range of butyric acid CF between 2.6-3.2. Conversely, the HPRO membrane demonstrated a butyric acid CF of 4.0 at the cost of a longer concentration time of 300 min. Notably, the maximum butyric acid CFs were limited by the high initial osmotic pressure of the clarified fermentation broth, as indicated by a conductivity of 16.7 mS/cm. Near the end of the membrane concentration tests, the concentrated fermentation broth had an even higher ionic strength due to the membranes retaining and concentrating inorganic salts, as indicated by the conductivity range of 34.6-51.2 mS/cm and 59-100% elemental rejections quantified using ICP. The transmembrane pressure gradient, which is the solvent permeation driving force, thus approached zero with the increased osmotic pressure of the concentrated feed solution, leading to diminished membrane permeate flux. The observation of higher CF achieved with HPRO is consistent with the model that increased applied pressure can lead to a higher maximum product CF. It is also worth noting that the contribution of membrane fouling to the membrane permeate flux decline throughout the membrane product concentration tests was relatively low (1.8-5.7% for RO membranes, and 9.2% for the HPRO membrane) compared to the osmotic pressure increase (FIG. 5E).

Because of the increasing permeate butyric acid concentration throughout the fermentation broth concentration tests, it may be expected that there is a tradeoff between butyric acid CF and product recovery for a given membrane, a higher product CF may be achieved at the cost of lower product recovery (or higher product loss into the permeate), and vice versa (FIG. 5D). Due to the overall high butyric acid selectivity of all tested RO membranes, despite this CF-PR tradeoff, butyric acid PR remained above 90% at the end of all fermentation broth concentration tests with total permeate volume recovery in the range of 64.5-70.9% (FIG. 5C). Compared to the seven RO membranes, the HPRO membrane demonstrated the highest butyric acid CF of 4.0 with a butyric acid PR of 94.7% (FIG. 5C) with total permeate volume recovery of 76.0%, consistent with its superior butyric acid selectivity as indicated by the lowest butyric acid permeability coefficient of 1.4 L·m⁻²·h⁻¹.

It is also worth noting that for all tested RO and HPRO membranes, the concentrated fermentation broth demonstrated a solution pH of approximately 5.1 similar to 5.0 of the original feed, while the permeate samples showed a slightly lower pH of approximately 4.2. The deviation of pH for both membrane permeate and concentrate from the original feed pH can be explained by water dissociation on both sides of membrane to maintain the charge balance and water dissociation constant throughout membrane product concentration. A lower pH in membrane permeate than the feed indicated that all the tested RO and HPRO membranes preferentially transport H+ over OH—. As a result, at the end of membrane product concentration, a higher percentage of the butyrate that passed through the membrane was in the acid form, while the retained butyrate exhibited a slightly increased amount of the salt form. For example, 95.1% and 28.0% of the butyrate was in the acid form in HPRO permeate and retentate, respectively, indicating a much higher membrane rejection of 99.4% for butyrate in the acid form compared with 83.5% for butyrate in the salt form.

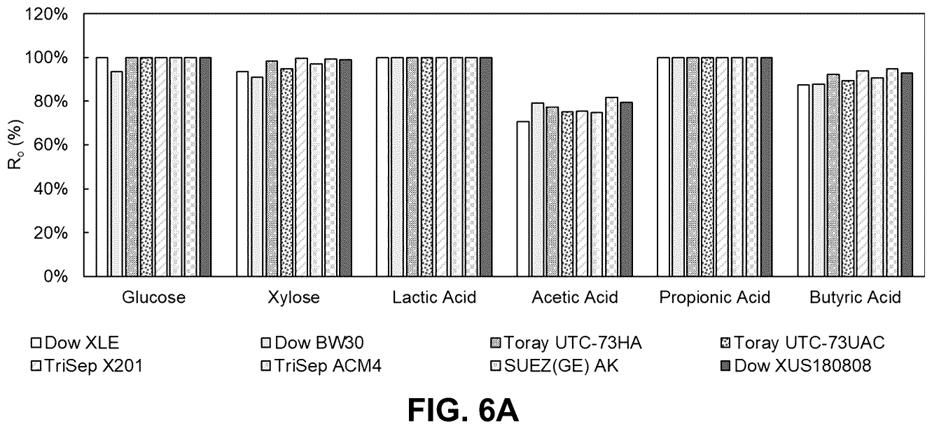

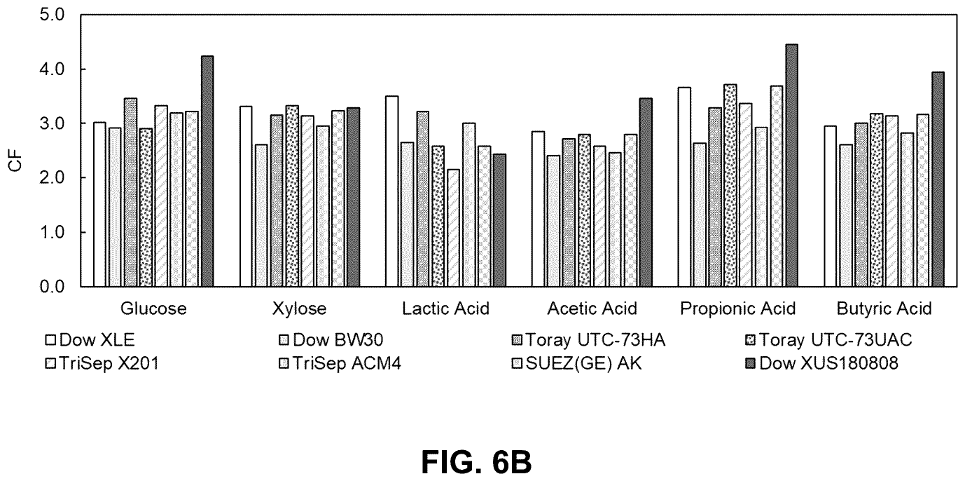

In addition to butyric acid, RO and HPRO membranes were tested for effectiveness concentrating other components in the fermentation broth, including glucose (measured CF range 2.9 4.2), xylose (CF: 2.6-3.3), lactic acid (CF: 2.1-3.5), acetic acid (CF: 2.6-4.4), and propionic acid (CF: 2.6 3.9) (FIGS. 6A and 6B). The highest CF for all the above fermentation broth components, was achieved with the HPRO membrane. It is notable that the collected RO and HPRO membrane permeate samples exhibited higher carboxylic acid purities (excluding water) than the clarified fermentation broth due to their 100% rejections of lactic acid and propionic acid (partly attributed to their presence in the fermentation broth with much lower concentrations), and >93.5% rejections of glucose and xylose. For example, the accumulated HPRO membrane permeate collected at the end of the fermentation broth concentration test had much higher contents of carboxylic acids, consisting of 36.8 wt % acetic acid, 50.4 wt % butyric acid, and 12.8 wt % xylose (FIG. 5F), as compared to 8.1 wt % acetic acid, 32.2 wt % butyric acid, and 56.4 wt % xylose in the original clarified fermentation broth. It was evident that the increase of acetic acid purity is even higher than butyric acid in HPRO membrane permeate, attributed to the higher membrane rejection and thus lower permeation of butyric acid due to its larger molecular size. Despite the observed increase in acetic acid and butyric acid purities, the use of RO and HPRO permeation alone for carboxylic acid purification from fermentation broth may yield relatively low product recovery in the permeate stream (only 17.1% for acetic acid and 5.3% for butyric acid). Instead, subsequent solvent extraction of carboxylic acids the RO and HPRO retentates may function as a more effective recovery approach.

The solvent extraction efficiency of butyric acid from the concentrated fermentation broth was subsequently evaluated with the tested RO and HPRO membranes, compared to the unconcentrated fermentation broth as a reference. The organic extractant mixture used in this study consisted of 20 vol % trioctylphosphine oxide, 40 vol % 2-undecanone, and 40 vol % mineral oil (light), which demonstrated an acceptable partition coefficient for butyric acid at pH 5.7. The solvent extraction experiments for the partition coefficient and extraction efficiency measurements were conducted in an overlay extraction setup with an aqueous-to-organic volume ratio of 1:1, with the intention to mimic a continuous solvent extraction unit in an ISPR process, such as a membrane-based emulsion separator (MBES) or a membrane contactor. Specifically, 3 mL of both aqueous and organic phases were added to 15 mL graduated tubes via two minutes of vortex mixing to accelerate mass transfer. Equilibrium was established by allowing the mixture to settle and phase separate at room temperature for 24 hours. The butyric acid partition coefficient and extraction efficiency were determined by measuring the butyric acid concentrations in the aqueous phase before and after liquid-liquid extraction (LLE) using HPLC.

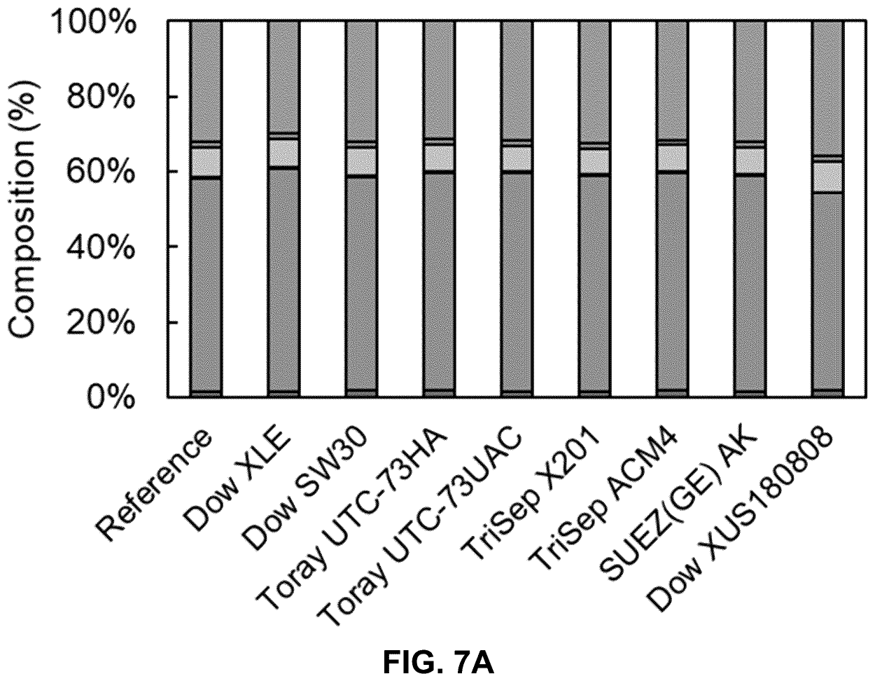

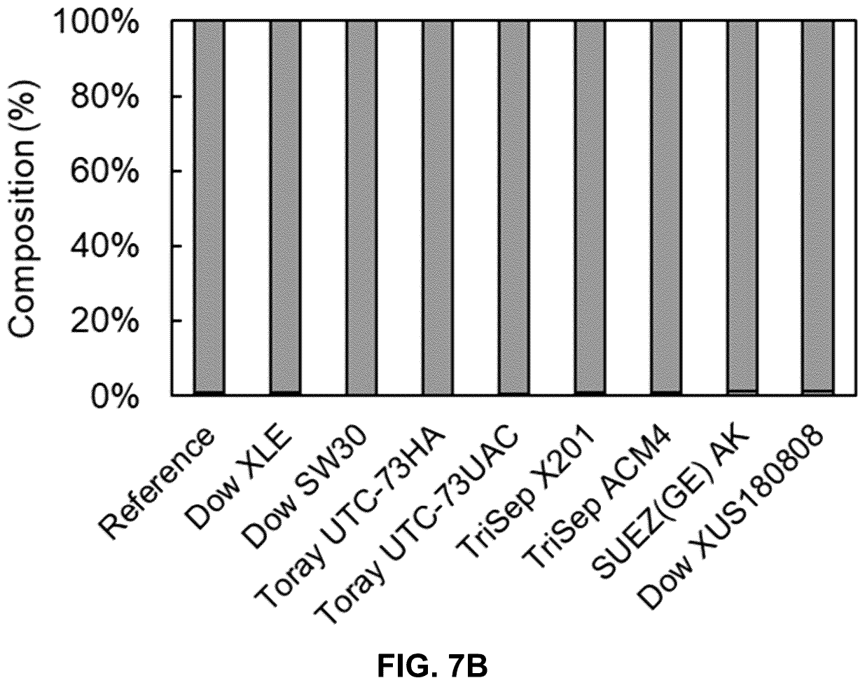

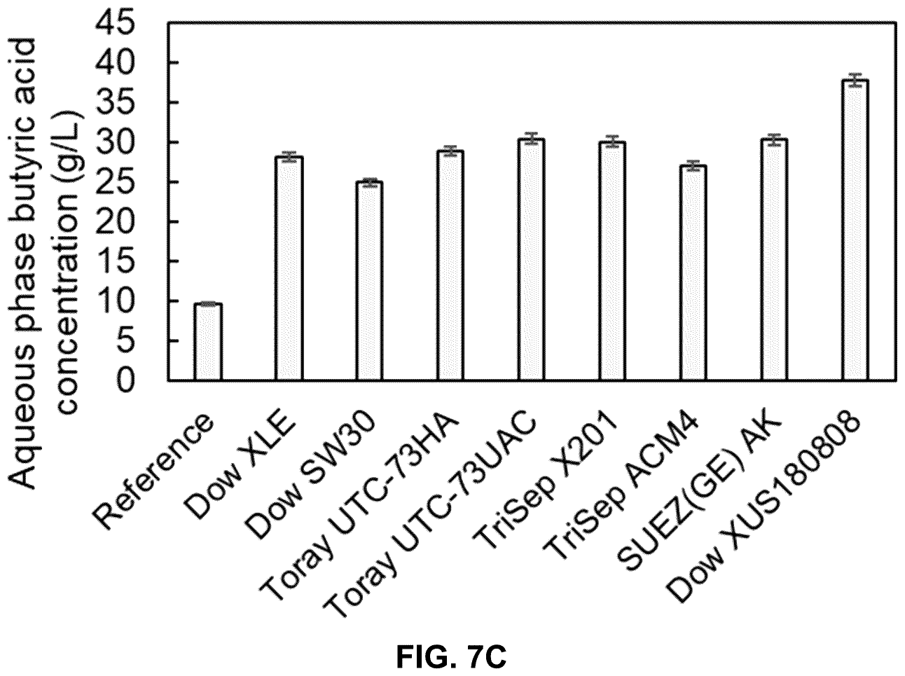

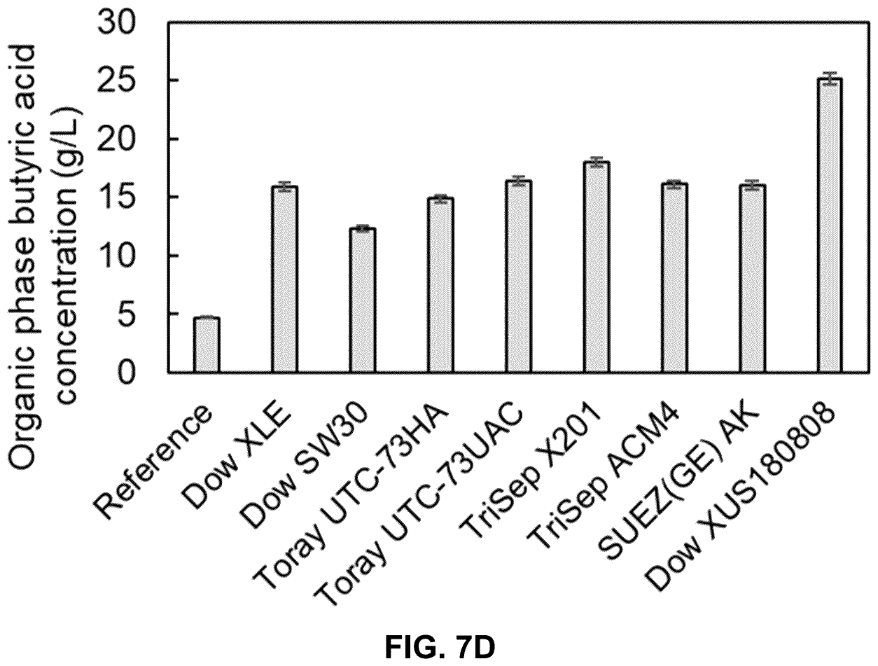

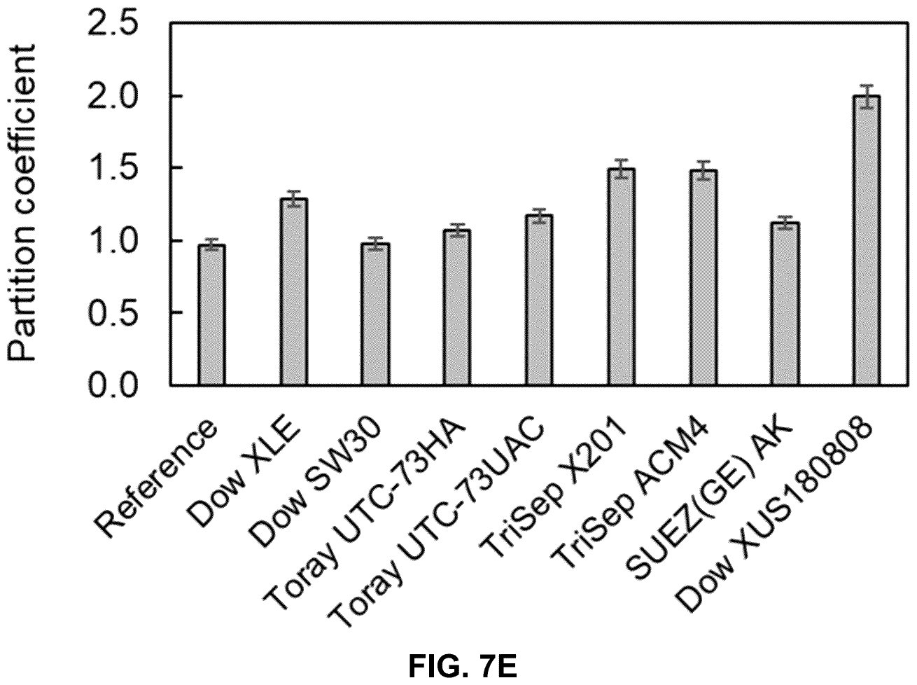

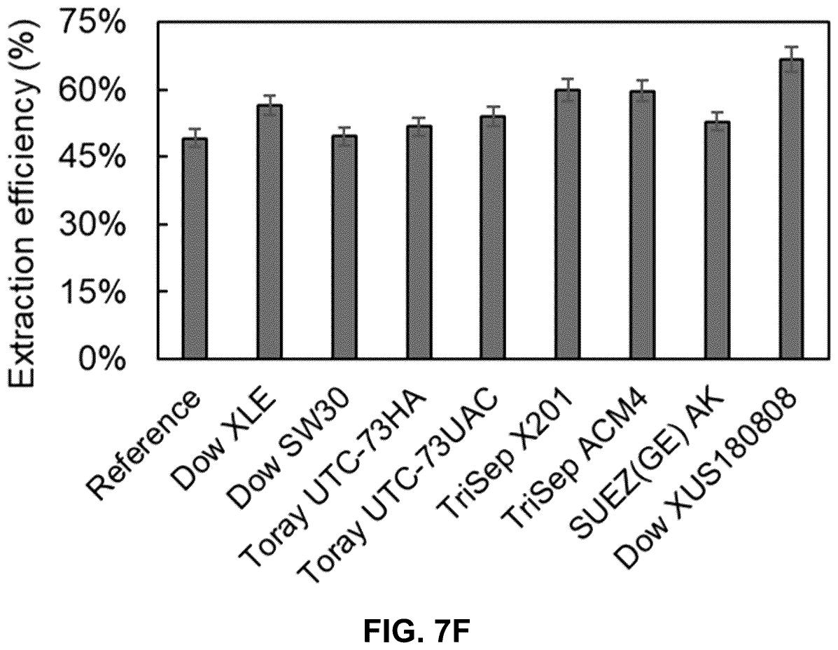

As verified by HPLC, although the RO and HPRO process concentrated both butyric acid and other components in the fermentation broth including glucose, xylose, and other shorter-chain carboxylic acids, LLE effectively purified carboxylic acids from this complex mixture via selective extraction (FIGS. 7A and 7B; Note that solvent extraction was carried out with organic extractant mixture consisting of 20 vol % trioctylphosphine oxide, 40 vol % 2-undecanone and 40 vol % mineral oil (light), and an aqueous-to-organic volume ratio of 1:1). The selected organic solvent mixture demonstrated no extraction of glucose, xylose, and propionic acid, and extraction efficiency (EE) of only up to 19.4% and 3.4% for lactic acid and acetic acid, respectively, which were significantly lower compared to 49.2-66.6% for butyric acid. Consequently, excluding the solvent, the organic phase consisted of 100 wt % carboxylic acids, of which 98.9 wt % is butyric acid. Comparatively, the clarified and membrane concentrated fermentation broth (i.e., aqueous stream before LLE) only contained 39.3-45.8% carboxylic acids, in which 30.0-35.9% was butyric acid. The effective purification of carboxylic acids, especially butyric acid, is particularly beneficial for ISPR, where the raffinate stream after LLE contains mostly glucose and xylose, which would be sent back to the bioreactor in an integrated process. The preferred extraction of butyric acid by the selected organic solvent allows the shorter-chain carboxylic acids, such as lactic acid, acetic acid, and propionic acid, to also return to the bioreactor after LLE, which can be further chain-elongated to improve overall butyric acid yields. The membrane-based concentration step also demonstrated significant impacts on the subsequent solvent extraction efficiency. As discussed above, RO and HPRO concentration led to increased butyric acid concentration in the concentrated C. tyrobutyricum fermentation broth to 24.9-30.4 g/L and 37.7 g/L, respectively, relative to 9.6 g/L in the originally clarified fermentation broth (FIG. 7C). This increase in butyric acid concentration in the aqueous phase was also reflected in its organic phase concentration after LLE. Indeed, after the partitioning equilibrium was reached, the butyric acid concentrations in the organic solvent were determined to be 12.3-18.0 g/L and 25.1 g/L with RO and HPRO concentration of the fermentation broth, respectively, relative to only 4.7 g/L for the reference (or ‘base’) scenario without membrane concentration (FIG. 7D). The butyric acid CF values in the organic phase, ranging from 2.6-5.3, were even higher compared to the aqueous phase of 2.6-4.0 (FIG. 5C). The higher butyric acid CF values in the organic phase relative to the aqueous phase are attributed to greater solvent partition coefficients and thus butyric acid extraction efficiencies. Indeed, solvent partition coefficients for the RO concentrated fermentation broth were determined to be within the range of 1.0-1.5, compared to that for the original clarified fermentation broth of 1.0 (FIG. 7E). The higher solvent partition coefficients led to higher butyric acid extraction efficiencies for the RO concentrated fermentation broth, ranging from 49.6-59.9%, compared to 49.2% for the original clarified fermentation broth. As expected, the HPRO concentrated fermentation broth demonstrated an even higher partition coefficient and extraction efficiency of 2.0 and 66.6%, respectively (FIGS. 7E and 7F). The observed increase in solvent partition coefficient for membrane concentrated fermentation broth may be due to the combined effects of the higher butyric acid concentration in the aqueous stream and thus greater partitioning driving force, and the differences in concentrated broth pH due to different membrane H+ rejections. Moreover, the integration of the RO and HPRO membrane concentration into ISPR process also led to significantly reduced solvent usage. Membrane concentration of butyric acid as well as other components in the fermentation broth was achieved by letting through (and thus removing) water into the permeate. Subsequently, the concentrated fermentation broth with reduced volume was sent for solvent selective extraction. For the same LLE organic-to-aqueous phase volume ratio of 1:1, RO membrane concentration can lead to 64.5-70.9% solvent usage reduction, and HPRO membrane concentration can lead to an even higher reduction of 76.0%. The reduced solvent usage leads to lower process cost and enhanced process environmental impacts, demonstrated below.

These modeling and experimental measurements identified the major factors limiting the maximum achievable concentration factor (CF) of 4.0 for butyric acid concentration with a HPRO membrane, compared to 2.6-3.2 for conventional reverse osmosis (RO) membranes. The resulting concentrated aqueous stream underwent liquid-liquid extraction with an organic solvent and distillation for butyric acid purification and solvent recycle. The integration of HPRO product concentration into an in-situ product recovery (ISPR) process leading to >5-fold increase in the final butyric acid concentration in the organic phase, and a concomitant 76% reduction in organic solvent usage. These improvements led to an estimated 53% and 46% reduction in ISPR butyric acid production cost and greenhouse gas (GHG) emissions, respectively, considerably exceeding the process performance when integrating conventional RO product concentration. Overall, the integration of an HPRO membrane for product concentration enables more economical and sustainable bioproduct recovery from dilute aqueous streams.

### Example 2: Membrane-Based Emulsion Separator for Bioproduct Recovery

To increase carboxylic acid flux in continuous liquid-liquid extraction (LLE), the use of a membrane-based emulsion separator (MBES) is an alternative approach to a membrane contactor. MBES-assisted LLE starts with sufficient mixing of the two phases to promote emulsion formation, which may be affected by a phase mixer. Emulsification ensures ample contact between the two phases through increased effective interfacial area and thus enhances the partitioning rate of a target product into the extraction solvent. After achieving equilibrium, the MBES, a porous membrane-based system, can be used to continuously separate the emulsion into a product-rich organic solvent and product-lean aqueous phase. Such complete in-line phase separation can be achieved via MBES systems by leveraging surface tension and fine-tuning pressure differential suggesting that MBES systems are promising for bioprocessing applications.

This example examines the use of an MBES system in place of a membrane contactor. Based on the potential of membrane demulsification systems in bioprocessing applications, an MBES unit operation was evaluated to determine if it could improve the efficiency and economics of the continuous LLE-based solvent extraction of biochemicals created in biosynthesis processes, such as fermentation. To that end, a short-chain carboxylic acid, butyric acid, produced via fermentation with Clostridium tyrobutyricum was used as a case study to evaluate the technical feasibility of an MBES-assisted LLE process. The goal was to optimize the extraction rate, membrane throughput, and phase separation efficiency of the MBES unit operation. The optimized conditions were used to evaluate membrane fouling propensity, and the feasibility of utilizing fermentation broth pre-filtration and membrane backwashing to mitigate fouling. Following process optimization, the economics and greenhouse gas (GHG) emissions of an MBES-assisted LLE process with the membrane contactor-assisted LLE process were compared.

To evaluate the performance of MBES-assisted LLE for solvent extraction of butyric acid at different permeate fluxes and phase volume ratios, a solution of butyric acid at 10 g/L and pH 5 was prepared as the ‘mock broth’ representing a single component model solution to eventually mimic the real Clostridium tyrobutyricum fermentation broth. 10 g/L as a target butyric acid titer in a bioreactor at pH 5 is able to minimize product inhibition for the bacterium while creating a sufficient driving force for partitioning into the organic phase in LLE. For these initial experiments, an organic extractant mixture consisting of 70 vol % Cyanex 923 and 30 vol % mineral oil was used, which exhibits the optimal partition coefficient for butyric acid at pH 5.3. Cyanex 923 is a commercial mixture of phosphine oxides.

The aqueous and organic phases were mixed in a range of phase volume ratios using a magnetic stir plate to form emulsions to mimic an in-line phase mixer and maximize the contact area between the aqueous and organic phases. The emulsion was then pumped into the hydrophobic polytetrafluoroethylene (PTFE) membrane-based MBES unit (SEP-200 and OB-2000-S200F; pore size approximately 1 μm and effective membrane area approximately 60 cm²; Zaiput Flow Technologies) at a range of feed flow rates (with different corresponding permeate fluxes) for membrane-based demulsification. Feeding this emulsified stream to the MBES unit resulted in the continuous separation of a butyric acid-lean aqueous and butyric acid-rich organic phases. The phase separation efficiency (PS) of the MBES system was evaluated by measuring the water contents in the feed and permeate streams using Karl Fisher (KF) titration and compared with the conventional overlay LLE as a reference using the same organic solvent composition. There exists a thermodynamic limit of the phase separation efficiency at each aqueous:organic phase volume ratio due to co-extraction of water. For example, the maximum achievable PS is 93.1% at phase volume ratio of 1.0 and 94.9% at phase volume ratio of 2.0, which is also the thermodynamic limit of PS.

Since membrane demulsification using the MBES system is based on the differences in membrane affinity of the different solvents, membrane surface hydrophilicity is an important indicator of its phase separation efficiency. An increase in the membrane surface hydrophilicity often indicates that the membrane surface is wetted by the retained phase and thus is often correlated with a compromised membrane phase separation efficiency.

Butyric acid extraction efficiency was also evaluated using the MBES system by measuring the butyric acid concentrations in the aqueous phase before and after the MBES-assisted LLE process using high-performance liquid chromatography (HPLC), and compared that to the previously reported results of a membrane contactor-assisted LLE experiment as the reference, again using the same organic solvent composition.

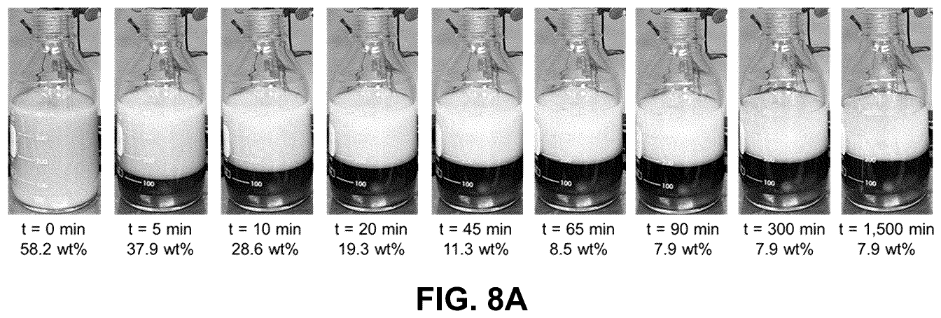

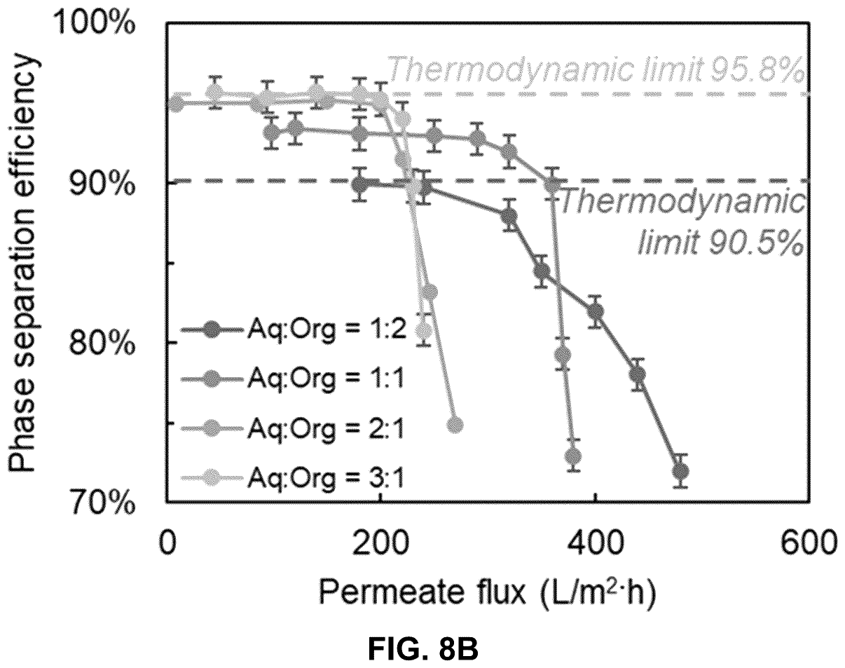

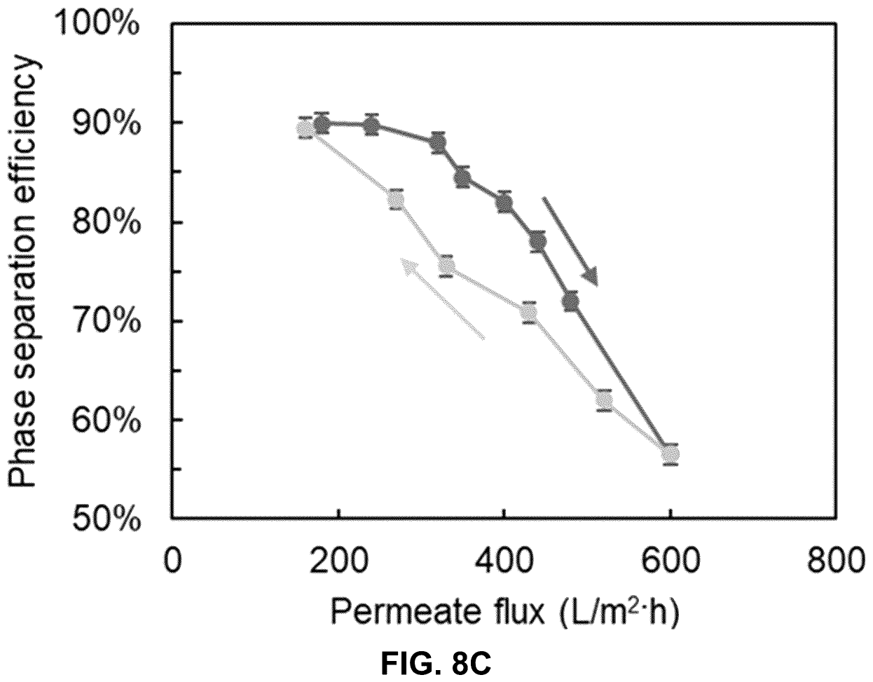

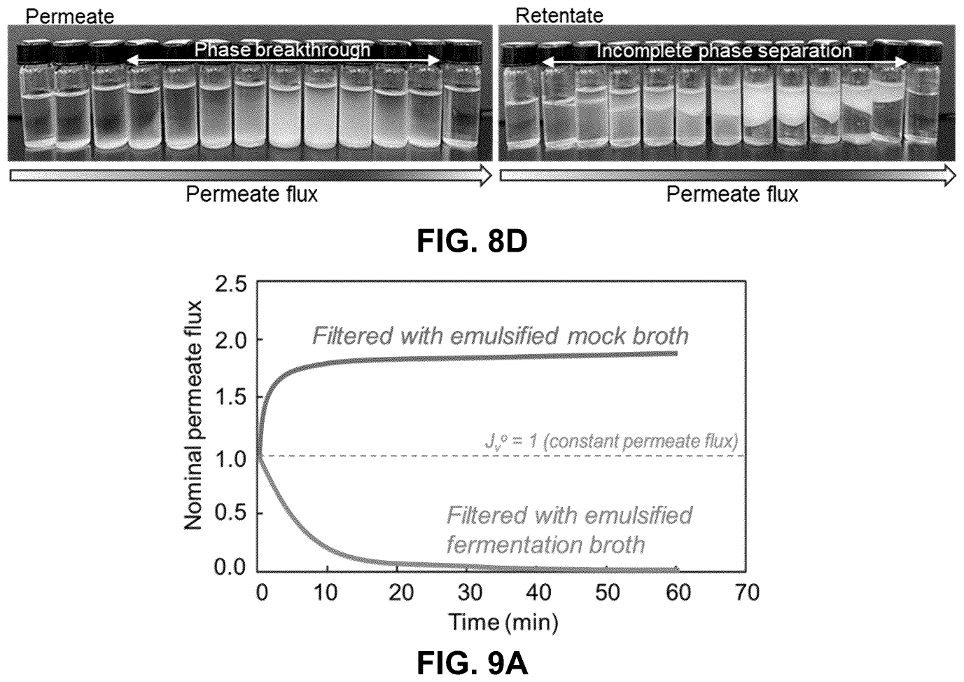

From the initial phase mixing experiments, it was determined that it only took three minutes to reach the butyric acid partition equilibrium (with a measured partition coefficient KD equal to 1) after a vigorous phase mixing (at a phase volume ratio of 1:1 and a stir rate of 650 rpm). The vigorous phase mixing results in emulsion formed with the aqueous phase present in the form of droplets with an average diameter of approximately 3.5 μm (measured using a Zetasizer) dispersed in the continuous organic phase and thus allowed droplet-based liquid-liquid microextraction with accelerated partitioning. However, despite the significant increase in the partitioning rate, the experimentally formed emulsion is kinetically stable and unbreakable via natural separation in overlay LLE. As shown, the top organic layer was still in the form of an emulsion with 7.9 wt % water content after 1,500 minutes settling time via solvent overlay separation (FIG. 8A). The impractically long time required for natural phase separation is attributed to the presence of a robust oil/water interface, making the solvent extraction via a direct overlay infeasible. It may be possible to shorten this time for natural phase separation by the addition of a surfactant. Conversely, the complete phase separation achieved by the MBES can dramatically accelerate the demulsification rate; specifically, it required only 10 min for the MBES (at its maximum permeate flux with complete phase separation; FIG. 8B) to continuously separate a 400 mL emulsion. Notably, the membrane demulsification time with the MBES system could be further reduced with a larger membrane area than 60 cm² used in the present example. To accelerate and achieve complete demulsification, the MBES unit was used to separate the emulsion into a butyric acid-lean aqueous phase and a butyric acid-rich organic phase (FIG. 8B). Due to the high surface hydrophobicity of the chosen membrane, it was expected that the organic phase would permeate through the membrane, while the retentate would comprise the aqueous phase due to the differences in membrane solvent affinity (i.e., wettability) and preferential transport. The operating conditions for the continuous membrane demulsification using MBES were:
Effective total membrane area of MBES: 60 cm²
Mock fermentation broth concentration: 10 g/L butyric acid
Mock fermentation broth: pH 5
Phase separation efficiency: 50% (at an aqueous:organic phase volume ratio=1)
Range of Karl Fisher titration measurements of water content in the organic solvent phase: 58.2 wt %-7.9 wt %.
Membrane permeate flux: 290.0 L/m²·h
MBES phase separation efficiency (PS): 70-100%
Thermodynamic limits of PS (at an aqueous:organic phase volume ratio 0.5, 1.0, 2.0, and 3.0): 90.5%, 93.1%, 94.9%, and 95.8% (respectively)
Aqueous:organic phase volume ratio 1:2-3:1
Overall butyric acid flux: 1,450.0 g/m²·h

The MBES unit achieved complete phase separation at a lower range of permeate fluxes for various phase volume ratios (FIG. 8B). For example, the MBES system enabled complete separations of an emulsion at an aqueous:organic phase volume ratio of 1.0 into a pure aqueous phase as the retentate (water content equal to 100 wt %) and an organic phase as the permeate with water content at the thermodynamic limit for permeate flux values <290.0 L/m²·h. At a higher range of permeate flux, membrane phase breakthrough (i.e., intrusion) was observed (FIGS. 8B-8D). At these higher permeate fluxes, the aqueous phase wetted the hydrophobic PTFE membrane, resulting in increased surface hydrophilicity (with 40.9% reduced membrane surface water contact angle, and surface free energy of hydration decreased from −30.8 to −92.1 mJ/m²), greater aqueous phase-membrane affinity, and thus up to 28.0% aqueous phase intrusion (FIG. 8B). It is noted that the increased preferential transport of the aqueous phase also led to membrane rejection of the organic phase, resulting in both permeate and retentate streams composed of mixtures of aqueous and organic phases.

The compromised membrane phase separation efficiency at higher permeate flux values can be 100% reversed by reducing the permeate flux (FIG. 8C). However, the reversal curves (in the direction of decreasing permeate flux) of the permeate and retentate water content do not perfectly overlay with the original ones in the direction of increasing permeate flux), due to the interplay between membrane surface wetting and phase breakthrough. This is because a more wetted membrane exhibits lower transport resistance to the aqueous phase, which further increases the potential of aqueous phase breakthrough and surface wetting in a positive feedback loop. Thus, a lower permeate flux was needed to achieve the same membrane phase separation efficiency after membrane surface and/or pore wetting.

At an aqueous:organic phase volume ratio equal to 1.0, the MBES butyric acid extraction efficiency (EE) was calculated to be 50%. The butyric acid extraction efficiency was limited by the organic solvent partition coefficient (KD) and aqueous:organic phase volume ratio but can be improved with a counter-current multi-stage membrane system. With a membrane permeate flux of 290.0 L/m²·h, the overall butyric acid flux of the present single-stage MBES system was calculated to be 1,450.0 g/m²·h, which is substantially higher than 8.9 g/m²·h achievable with a membrane contactor (as shown in Table 3-Comparison of the butyric acid extraction rate between an MBES system and a membrane contactor unit operation). The butyric acid extraction rate was measured using 70 vol % Cyanex 923, 30 vol % mineral oil to extract butyric acid from 10 g/L butyric acid aqueous mock solution at pH 5 and a phase volume ratio of 1. Two Liqui-Cel Extra-Flow 2.5×8 membrane contactor units (3M) were used for membrane contactor LLE. The aqueous and organic phases were continuously circulated through the lumen and shell sides of the membrane contactor, respectively, at the flow rate of 40 mL/min. A membrane contactor can achieve butyric acid extraction efficiency as high as the thermodynamic limit (partition equilibrium) according to a previous study (P. O. Saboe, et al., In situ recovery of bio-based carboxylic acids. Green Chemistry, 2018, 20:1791-1804.).

TABLE 3

| | Membrane | Butyric acid | Butyric | Membrane area needed |
|---|---|---|---|---|
| | area | extraction | acid flux | (to extract 100 g/h |
| | (m²) | rate (g/h) | (g/m² · h) | butyric acid) (m²) |
| MBES | 0.006 | 8.4 | 1,450.0 | 0.07 |
| Membrane | 1.4 | 12.5 | 8.9 | 11.2 |
| contactor | | | | |

As a result, to have the same butyric acid extraction rate, an MBES would require an approximately 160-fold smaller membrane area than a membrane contactor. Notably, this calculated butyric acid flux will change as a function of phase volume ratio. Here, an aqueous:organic phase volume ratio equal to 1.0 was selected for a direct comparison with previous MC-assisted LLE results.

These results suggest considerable advantages in using membrane demulsification for butyric acid separations, based on the use of a mock broth solution containing only butyric acid at 10 g/L in water at pH 5. Importantly, realistic fermentation broth contains impurities including cell debris, thus making membrane fouling a potential consideration for the use of an MBES system, which can lead to both flux decline and compromised permeate quality. Thus, it was an objective to evaluate membrane fouling propensity over a 1-hour filtration with an emulsified feed stream prepared from the actual fermentation broth, relative to the emulsified mock broth described above.

The emulsified fermentation broth was prepared by filtering Clostridium tyrobutyricum fermentation broth using a rotating microfiltration (MF) ceramic disc (with pore size of 0.2 μm) and mixing 300 mL of the filtered fermentation broth with 150 mL organic phase, and again, an emulsified mock broth was prepared by mixing 300 mL 10 g/L butyric acid aqueous solution with 150 mL organic phase, both at a stir rate of 650 rpm for 3 minutes to achieve an aqueous:organic phase volume ratio equal to 2 for both systems. It was expected that by increasing the aqueous:organic phase volume ratio, one could decrease the volume of organic solvent used in the process and thus make the separation process more efficient.

Membrane fouling propensity is indicated by the normalized membrane permeate flux and flux decline, and the change in water content in the feed, permeate, and retentate streams. The flux decline caused by membrane fouling is often attributed to membrane surface cake layer buildup and membrane pore narrowing. The increase in membrane resistance for solvent permeation after membrane fouling is referred to fouling resistance.

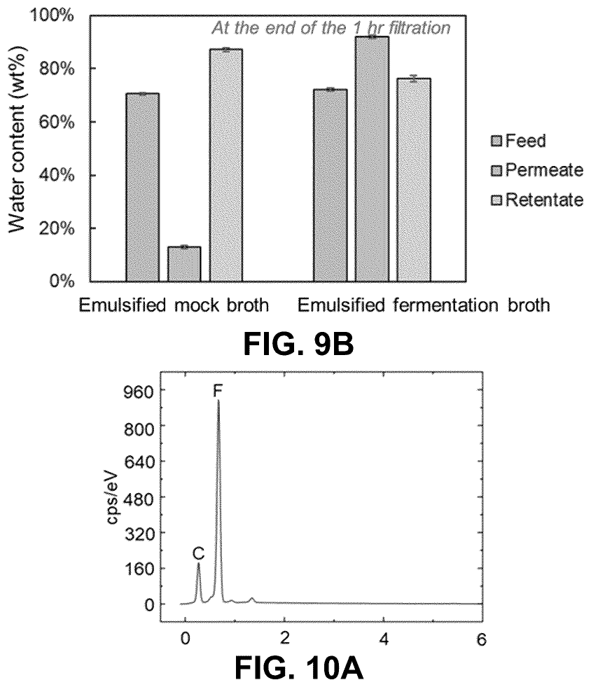

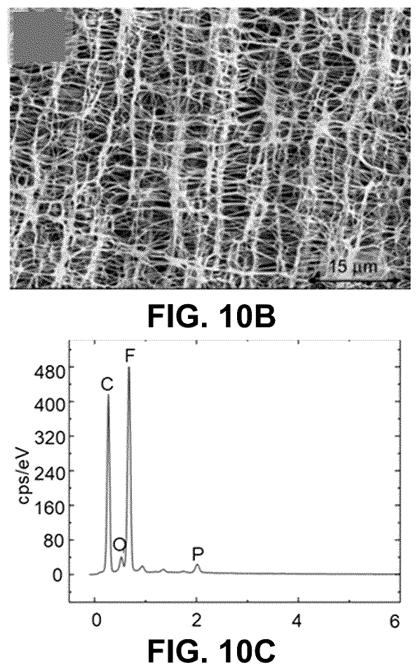

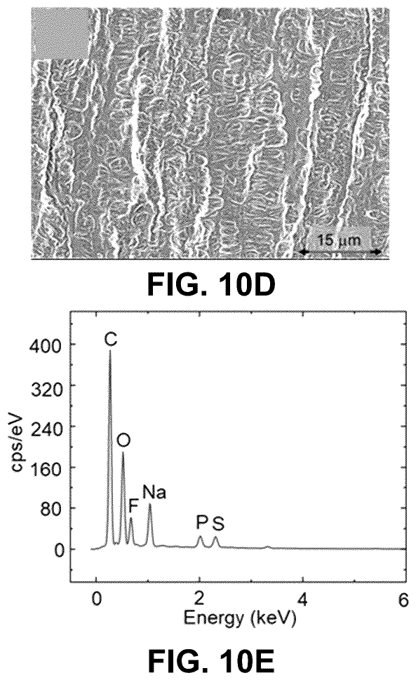

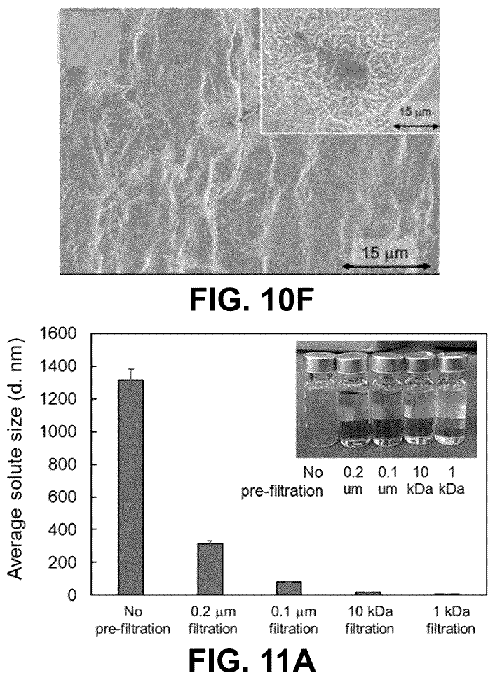

After a 1-hour membrane filtration experiment with the emulsified fermentation broth (at a feed flowrate approximately 29.5 mL/min), an approximately 96.1% flux decline was observed for the hydrophobic PTFE membrane (FIG. 9A). The reduced membrane permeate flux can be explained by the accumulation of foulants on the membrane surface, which served as a resistance layer (with a calculated fouling resistance of 3.0×1012 m⁻¹) and thus decreased organic solvent permeability through the membrane. Indeed, compared to the pristine membrane, a thick cake layer on the membrane surface was confirmed via scanning electron microscope images for fouled membrane surfaces (FIGS. 10B, 10D, 10F). As the Clostridium tyrobutyricum fermentation broth was pre-filtered by a 0.2 μm MF ceramic disk to remove all the intact cells before feeding into the MBES, only organic fouling should occur on the membrane surface and/or inside the pores. The organic fouling was also indicated by the energy-dispersive X-ray spectroscopy analysis showing the fouled hydrophobic PTFE membrane surface exhibited a 12.5-fold higher C/F ratio relative to the pristine membrane and O presence of 24 wt % (FIGS. 10A, 10C, 10E, Table 4). In Table 4, emulsion A is the emulsified mock broth. The membrane filtration was carried out at a feed flow rate of 140 mL/min for 1 hour and membrane phase breakthrough was observed. Emulsion B is the emulsified fermentation broth. Notably, even with a 96.1% flux decline, the MBES still exhibited approximately 6.2 times higher butyric acid flux than the membrane contactors. Table 4. Elemental composition of the different membrane surfaces as characterized using energy-dispersive X-ray spectroscopy.

TABLE 4

| Membrane | F | C | O | C/F | O/F |
|---|---|---|---|---|---|
| Pristine hydrophobic | 79.1% | 20.9% | — | 0.26 | — |
| PTFE membrane | | | | | |
| Hydrophobic PTFE membrane | 50.6% | 42.5% | 2.1% | 0.84 | 0.04 |
| filtered with emulsion A | | | | | |
| Hydrophobic PTFE membrane | 14.2% | 46.1% | 23.6% | 3.25 | 1.67 |
| filtered with emulsion B | | | | | |

Additionally, the foulant layer buildup on the membrane surface not only caused membrane flux decline but also led to aqueous phase breakthrough (FIG. 9B). Indeed, unlike the pristine hydrophobic PTFE membrane, which separates almost pure organic phase (3.6 wt % water content) as the permeate, the fouled membrane has a permeate stream composed of 92.0 wt % aqueous and 8.0 wt % organic phases (FIG. 9B). The observed phase breakthrough is attributed to the reduced hydrophobic PTFE membrane surface hydrophobicity (as indicated by the decrease in surface free energy of hydration from −30.8 to −108.4 mJ/m²) with the accumulated fouling layer. Membrane surface wetting thus altered the solvent-membrane surface interactions by increasing the aqueous phase membrane affinity, leading to preferred transport of the aqueous phase.

It may be argued that the observed high fouling propensity is expected due to the hydrophobic nature of the tested membrane, which can lead to the hydrophobic interactions (i.e., dispersive forces) between membrane surface and foulant molecules. Hydrophilic membranes, on the other hand, could exhibit much lower fouling propensity due to the high surface hydrophilicity which reduce the hydrophobic interactions and thus mitigate surface adsorption or deposition. Therefore, the commercial hydrophilic PTFE membrane from Zaiput Flow Technologies (IL-2000-S200F; pore size ˜1 μm) was tested for demulsification of the emulsified fermentation broth, which should result in the aqueous phase as the permeate while retaining the organic phase. It was observed that the hydrophilic PTFE membrane, which has much a higher surface hydrophilicity (52.2% reduced water SD contact angle compared to the hydrophobic membrane), exhibited lower fouling propensity as indicated by a 62.5% flux decline over 1 hour filtration, which is significantly less than the 96% flux decrease for the hydrophobic PTFE membrane. The lower fouling propensity of the hydrophilic membrane was consistent with a thinner cake layer observed on the fouled hydrophilic membrane surfaces (with some membrane pores and void fractions exposed) via scanning electron microscope images and a relatively minor increase in the surface C/F and O/F ratios compared to the fouled hydrophobic PTFE membrane. Moreover, no phase breakthrough was observed for the hydrophilic PTFE membrane, but rather the permeate stream was always pure aqueous phase even at the end of 1 hour fouling tests. The preferred permeation of the aqueous phase throughout the fouling test was due to further increased surface hydrophilicity (and thus membrane affinity to the aqueous phase) after organic fouling (as indicated by surface free energy of hydration decreased from −109.1 to −125.6 mJ/m²). Despite this, the hydrophilic membrane is not suitable for separating emulsified fermentation broth (which is a water-in-oil emulsion) due to its significantly lower permeate flux. Indeed, the initial permeate flux of the hydrophilic PTFE membrane was only 4.0 L/m²·hr, which is 95.6% lower than that of the hydrophobic PTFE membrane. Such a low initial permeate flux is likely attributed to the limited contact area between the dispersive aqueous phase droplets and the membrane surface.

It is also interesting to note that, unlike the hydrophilic membrane, oil wetting (i.e., oil fouling) is not a concern for hydrophobic membrane separation of the water-in-oil emulsion. Even though the organic phase attachment does alter the membrane surface morphology, as indicated via the scanning electron microscope images (by filling in all the pores and void fractions of the membrane; FIG. 10D) and membrane surface chemical composition as indicated by the energy-dispersive X-ray spectroscopy results (with increased C/F and O/F ratios; FIGS. 10A-10F), it does not lead to membrane permeate flux decline. Instead, an 85.7% increase in hydrophobic PTFE membrane permeate flux was observed at the end of 1 hour of membrane filtration with the emulsified mock broth, which can be explained by the absorption of organic phase into the membrane matrix (FIG. 10D), leading to membrane solvation (i.e., swelling).

To address the decreased membrane performance caused by membrane fouling, membrane fouling mitigation strategies were evaluated. Protein is believed to be the dominant foulant in the emulsified fermentation broth solution with a total concentration as high as 45.1 g/L relative to 18.4 mg/L of DNA in the fermentation broth pre-filtered with 0.2 μm MF membrane disc, measured using Thermo Scientific™ BCA Protein Assay and DNeasy PowerSoil Pro Kit, respectively.

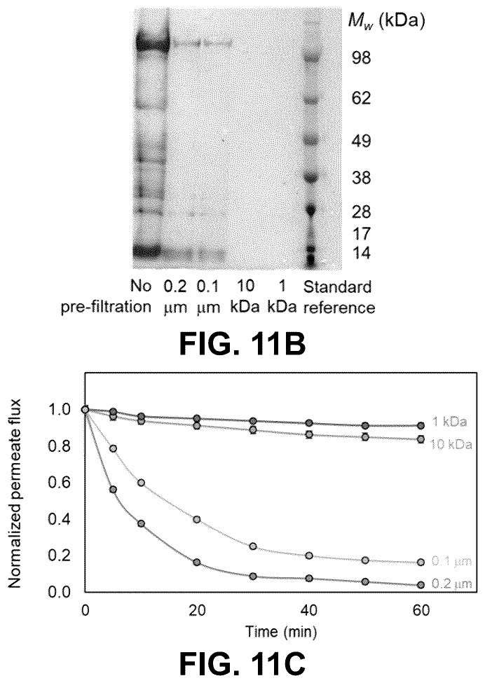

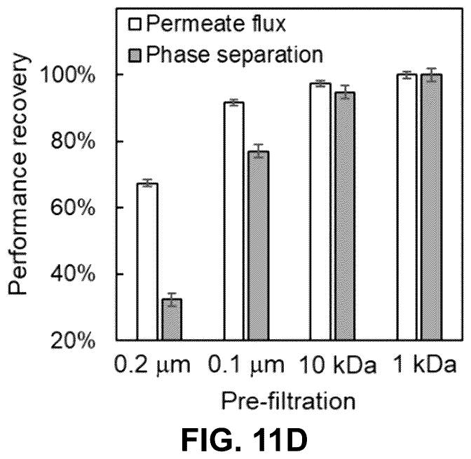

The effectiveness of MF membranes (with pore sizes of 0.2 and 0.1 μm; GNWP04700 and VCTP04700, Millipore Sigma, Burlington, MA) and ultrafiltration (UF) membranes (with molecular weight cutoff values of 10 and 1 kDa; PLGC04310 and PLAC04310, Millipore Sigma, Burlington, MA) filtration for protein removal from Clostridium tyrobutyricum fermentation broth was evaluated first. The above MF and UF membrane pre-filtrations of fermentation broth were carried out with a 50 mL dead-end stirred UF cell (Amicon 8050, Millipore Corporation, Burlington, MA) at 3.4 bar. The effectiveness of protein removal using MF and UF membranes with different pore sizes was indicated by the average solute size (measured by Zetasizer) and the results of protein gel electrophoresis measured for the membrane permeate as compared to the unfiltered Clostridium tyrobutyricum fermentation broth. The MBES performance was then evaluated in a 1-hour filtration experiment using the emulsified, pre-filtered fermentation broth in terms of both flux decline and water content in the final permeate and retentate streams. After the filtration test, the fouled membranes were cleaned with a two minute membrane permeate backwash to evaluate membrane cleaning. The performance recovery was determined for both permeate flux and phase separation efficiency. For MF and UF membrane filtration of the Clostridium tyrobutyricum fermentation broth for protein removal, it was observed that the filtered fermentation broth exhibited a progressively lighter color at higher resolution of filtration, which was consistent with the reduced average solute particle size as a function of filter cutoff (FIG. 11A). The effectiveness of membrane protein removal was also demonstrated by protein gel electrophoresis, where 100% removal of larger proteins (>14 kDa) was achieved with both 10 kDa and 1 kDa UF membranes (FIG. 11B).

MF and UF membrane pre-filtration of the Clostridium tyrobutyricum fermentation broth demonstrated effective protein removal and thus lead to significantly reduced MBES membrane fouling propensity. Over 1 hour filtration of emulsified pre-filtered fermentation broth (filtered with membranes of different pore sizes), the hydrophobic PTFE membrane fouling propensity was lowered by 12.5%, 80.0%, and 87.5% (FIG. 11C) using membranes with pore size of 0.1 μm, 10 kDa, and 1 kDa, respectively, relative to 0.2 μm MF pre-filtration. Moreover, phase breakthrough in the permeate was also minimized with membrane protein removal, as indicated by the final permeate water content measurement lowered by up to 63.0%. For membrane performance recovery, a 2 minute membrane backwash (where the direction of the permeate flow is reversed to dislodge foulants accumulated on the membrane surface or inside the pores) was carried out at the end of the 1 hour membrane filtration test, leading to 68.6-100.0% and 32.1-100.0% permeate flux and phase separation recovery (FIG. 11D). Especially the 1 kDa UF membrane pre-filtration exhibited complete recovery of both membrane permeate flux and phase separation efficiency indicating the absence of irreversible fouling, while the use of larger pore size membranes led to less effective protein removal and thus 2.6-32.6% irreversible fouling (Table 5).

Table 5. Summary of hydrophobic PTFE membrane intrinsic, reversible, and irreversible fouling resistance as affected by the pre-filtration MF and UF membrane pore size.

TABLE 5

| Pre-filtration | Rₘ (m⁻¹) | R_{fouling} (m⁻¹) | Rᵣₑᵥ (m⁻¹) | % Reversible | Rᵢᵣᵣₑᵥ (m⁻¹) | % Irreversible |
|---|---|---|---|---|---|---|
| 0.2 μm MF | 1.2 × 10¹¹ | 3.0 × 10¹² | 2.0 × 10¹² | 67.4% | 9.7 × 10¹¹ | 32.6% |
| 0.1 μm MF | 1.2 × 10¹¹ | 6.0 × 10¹¹ | 5.5 × 10¹¹ | 91.7% | 5.0 × 10¹⁰ | 8.3% |
| 10 kDa UF | 1.2 × 10¹¹ | 2.3 × 10¹⁰ | 2.2 × 10¹⁰ | 97.4% | 5.9 × 10⁸ | 2.6% |
| 1 kDa UF | 1.2 × 10¹¹ | 1.1 × 10¹⁰ | 1.1 × 10¹⁰ | 100.0% | 0.0 | 0.0% |

In Table 5, membrane intrinsic resistance (Rₘ), was determined from the relation Lₚ=1/μRₘ, where Lₚ is the membrane organic phase permeability, and μ is the organic solvent viscosity (0.0126 Pa's at 20° C.). Membrane permeability (Lₚ) was determined from the slope of a linear plot of membrane permeate flux as a function of the differences between applied pressure and intrusion pressure (i.e., Lₚ=Jᵥ/(ΔP−P_{I}), where Jᵥ is membrane permeate flux of the organic phase, ΔP is the applied transmembrane pressure, and P_{I} is the membrane organic phase intrusion pressure).

At the end of each fouling test, a determination was made of the membrane overall hydraulic resistance (R_{T}) being the sum of the intrinsic membrane resistance (Rₘ) and fouling resistance (R_{fouling}). It is noted the fouling resistance (R_{fouling}) is the sum of reversible and irreversible fouling resistances: R_{fouling}=Rᵣₑᵥ+Rᵢᵣᵣₑᵥ.

Membrane backwash with the permeate organic solvent was conducted by reversing the pump direction at approximately 20 mL/min for 2 min. The resistance of the backwashed membrane was then again determined with organic solvent, thereby allowing quantification of the combined intrinsic membrane and irreversible fouling resistances expressed as R′_{T}=Rₘ+Rᵢᵣᵣₑᵥ. Subsequently, Rᵣₑᵥ and Rᵢᵣᵣₑᵥ were determined given the calculated values of R_{T} and R′_{T}.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A method of extracting an organic compound from a media, comprising:
filtering an aqueous media comprising an organic compound through a high-pressure reverse osmosis (HPRO) membrane to concentrate the organic compound in an aqueous HPRO filtration retentate;
extracting the organic compound from the aqueous HPRO filtration retentate by a recovery process selected from the group consisting of liquid-liquid extraction, distillation, crystallization, electrodialysis, chromatography, adsorption, evaporation, and combinations thereof.

2. The method of claim 1, wherein
the organic compound is selected from the group consisting of alkanes, alkenes, alkynes, aromatic hydrocarbons, alcohols, phenols, ethers, aldehydes, ketones, carboxylic acids, esters, amides, acid anhydrides, amines, nitriles, alkyl halides, aryl halides, thiols, sulfides, sulfonic acids, phosphines, phosphates, and combinations thereof.

3. The method of claim 1, wherein
the organic compound is selected from the group consisting of acetic acid, propionic acid, butyric acid, valeric acid, caproic acid, lactic acid, and combinations thereof.

4. The method of claim 1, wherein
the HPRO membrane operates at a pressure between 800 psi and 1800 psi and achieves a concentration factor between 3.5 and 5.0 for the organic compound.

5. The method of claim 1, wherein
the HPRO membrane comprises a thin-film composite polyamide membrane.

6. The method of claim 1, wherein
extracting the organic compound from the aqueous HPRO filtration retentate comprises liquid-liquid extraction to produce an organic solvent comprising the organic compound.

7. The method of claim 6, wherein
the liquid-liquid extraction is membrane-based liquid-liquid extraction (MBLLE) comprising a membrane material selected from polyethylene, high density polyethylene, perfluoroalkylate, polytetrafluoroethylene, ultra-high molecular weight polyethylene, a copolymer of ethylene and tetrafluoroethylene, polyphthalamide, polyamide, polyester, polysulfone, polyethersulfone, cellulose, cellulose acetate, regenerated cellulose, and combinations thereof.

8. The method of claim 7, wherein
the MBLLE membrane material has a pore size between 2 μm and 0.1 μm.

9. The method of claim 6, wherein
the liquid-liquid extraction of the organic compound comprises extracting the organic compound into an organic phase with a solvent partition coefficient between 1.0 and 3.0.

10. The method of claim 6, wherein
the liquid-liquid extraction of the organic compound comprises extracting the organic compound into an organic phase with an extraction efficiency between 65% and 83%

11. The method of claim 6, further comprising
distilling the organic solvent comprising the organic compound to purify the organic compound from the organic solvent at a purity of between 90% and 100%.

12. The method of claim 1, wherein:
the HPRO retentate comprises a bioreactor reaction media and extracting the organic compound from the aqueous HPRO filtration retentate produces a spent HPRO retentate comprising a bioreactor reaction media.

13. The method of claim 11, further comprising:
returning the spent HPRO retentate comprising a bioreactor reaction media to a bioreactor to continuously remove the organic compound from the reaction media in a continuous biosynthetic production process.

14. A system for extracting an organic compound from a media, comprising:
a high-pressure reverse osmosis (HPRO) unit configured to accept an aqueous stream comprising an organic compound from a bioreactor and concentrate the organic compound in an aqueous HPRO filtration retentate; and,
an extraction unit, fluidly connected to the HPRO unit, configured to isolate the organic compound from the aqueous HPRO filtration retentate to form an isolated organic compound and a spent aqueous HPRO filtration retentate.

15. The system of claim 14, wherein
the extraction unit comprises a recovery process selected from the group consisting of liquid-liquid extraction, distillation, crystallization, electrodialysis, chromatography, adsorption, evaporation, and combinations thereof.

16. The system of claim 14, wherein
the organic compound is selected from the group consisting of alkanes, alkenes, alkynes, aromatic hydrocarbons, alcohols, phenols, ethers, aldehydes, ketones, carboxylic acids, esters, amides, acid anhydrides, amines, nitriles, alkyl halides, aryl halides, thiols, sulfides, sulfonic acids, phosphines, phosphates, and combinations thereof.

17. The system of claim 14, wherein
the HPRO unit comprises an HPRO membrane that operates at a pressure between 800 psi and 1800 psi, and achieves a concentration factor between 3.5 and 5.0 for the organic compound.

18. The system of claim 14, wherein
the extraction unit comprises a membrane-based liquid-liquid extraction (MBLLE) unit comprising a membrane material selected from polyethylene, high density polyethylene, perfluoroalkylate, polytetrafluoroethylene, ultra-high molecular weight polyethylene, a copolymer of ethylene and tetrafluoroethylene, polyphthalamide, polyamide, polyester, polysulfone, polyethersulfone, cellulose, cellulose acetate, regenerated cellulose, and combinations thereof.

19. The system of claim 18, further comprising
a first recirculation system for recycling spent aqueous HPRO filtration retentate from the MBLLE unit to a bioreactor.

20. The system of claim 19, further comprising
a second recirculation system for recycling an organic solvent from the extraction unit to be mixed with the HPRO filtration retentate.
